(19) United States
(12) Patent Application Publication
(10) Pub. No.: US 2020/0100795 A1
(43) Pub. Date: Apr. 2, 2020

(54) "Bowl-of-Spaghetti" Type Intrasacular Aneurysm Occlusion Device

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 16/693,267

(22) Filed: Nov. 23, 2019

Related U.S. Application Data
(63) Continuation-in-part of application No. 16/660,929, filed on Oct. 23, 2019.
Application No. 16/660,929 is a continuation-in-part of application No. 16/541,241, filed on Aug. 15, 2019.
Application No. 16/541,241 is a continuation-in-part of application No. 15/865,822, filed on Jan. 9, 2018.
Application No. 15/865,822 is a continuation-in-part of application No. 15/861,482, filed on Jan. 3, 2018.
Application No. 15/861,482 is a continuation-in-part of application No. 16/541,241, filed on Aug. 15, 2019.
Application No. 16/541,241 is a continuation-in-part of application No. 15/865,822, filed on Jan. 9, 2018.
Application No. 15/865,822 is a continuation-in-part of application No. 15/861,482, filed on Jan. 3, 2018.
Application No. 15/861,482 is a continuation-in-part of application No. 15/865,822, filed on Jan. 9, 2018.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12113; A61B 17/12118; A61B 2017/00292; A61B 17/1215; A61B 2017/12095; A61B 17/12172

(57) ABSTRACT

This invention is a “bowl-of-spaghetti” type intrasacular aneurysm occlusion device including: a catheter; embolic coils; a bowl or cup shaped neck bridge; an opening in the neck bridge through which the coils are inserted into the aneurysm sac; and a closure mechanism which closes the opening after the coils have been inserted into the aneurysm sac. It is a called a “bowl-of-spaghetti” type device because the neck bridge can look like a bowl and the embolic coils can look like spaghetti noodles in the bowl.

### CROSS-REFERENCE TO RELATED APPLICATIONS

This present application is a continuation in part of U.S. patent application Ser. No. 16/660,929 filed on 2019 Oct. 23. This present application also claims the priority benefit of U.S. provisional patent application No. 62/794,609 filed on 2019 Jan. 19. This present application also claims the priority benefit of U.S. provisional patent application No. 62/794,607 filed on 2019 Jan. 19. This present application also is a continuation in part of U.S. patent application Ser. No. 16/541,241 filed on 2019 Aug. 15. This present application also is a continuation in part of U.S. patent application Ser. No. 15/865,822 filed on 2018 Jan. 9. This present application also is a continuation in part of U.S. patent application Ser. No. 15/861,482 filed on 2018 Jan. 3.

U.S. patent application Ser. No. 16/660,929 claimed the priority benefit of U.S. provisional patent application No. 62/794,609 filed on 2019 Jan. 19. U.S. patent application Ser. No. 16/660,929 claimed the priority benefit of U.S. provisional patent application No. 62/794,607 filed on 2019 Jan. 19. U.S. patent application Ser. No. 16/660,929 was a continuation in part of U.S. patent application Ser. No. 16/541,241 filed on 2019 Aug. 15. U.S. patent application Ser. No. 16/660,929 was a continuation in part of U.S. patent application Ser. No. 15/865,822 filed on 2018 Jan. 9. U.S. patent application Ser. No. 16/660,929 was a continuation in part of U.S. patent application Ser. No. 15/861,482 filed on 2018 Jan. 3.

U.S. patent application Ser. No. 16/541,241 claimed the priority benefit of U.S. provisional patent application No. 62/794,609 filed on 2019 Jan. 19. U.S. patent application Ser. No. 16/541,241 claimed the priority benefit of U.S. provisional patent application No. 62/794,607 filed on 2019 Jan. 19. U.S. patent application Ser. No. 16/541,241 claimed the priority benefit of U.S. provisional patent application No. 62/720,173 filed on 2018 Aug. 21. U.S. patent application Ser. No. 16/541,241 was a continuation in part of U.S. patent application Ser. No. 15/865,822 filed on 2018 Jan. 9.

U.S. patent application Ser. No. 15/865,822 claimed the priority benefit of U.S. provisional patent application No. 62/589,754 filed on 2017 Nov. 22. U.S. patent application Ser. No. 15/865,822 claimed the priority benefit of U.S. provisional patent application No. 62/472,519 filed on 2017 Mar. 16. U.S. patent application Ser. No. 15/865,822 was a continuation in part of U.S. patent application Ser. No. 15/081,909 filed on 2016 Mar. 27. U.S. patent application Ser. No. 15/865,822 was a continuation in part of U.S. patent application Ser. No. 14/526,600 filed on 2014 Oct. 29.

U.S. patent application Ser. No. 15/861,482 claimed the priority benefit of U.S. provisional patent application Ser. No. 62/589,754 filed on 2017 Nov. 22. U.S. patent application Ser. No. 15/861,482 claimed the priority benefit of U.S. provisional patent application No. 62/472,519 filed on 2017 Mar. 16. U.S. patent application Ser. No. 15/861,482 claimed the priority benefit of U.S. provisional patent application No. 62/444,860 filed on 2017 Jan. 11. U.S. patent application Ser. No. 15/861,482 was a continuation in part of U.S. patent application Ser. No. 15/080,915 filed on 2016 Mar. 25 which issued as U.S. Pat. No. 10,028,747 on 2018 Jul. 24. U.S. patent application Ser. No. 15/861,482 was a continuation in part of U.S. patent application Ser. No. 14/526,600 filed on 2014 Oct. 29.

U.S. patent application Ser. No. 15/081,909 was a continuation in part of U.S. patent application Ser. No. 14/526,600 filed on 2014 Oct. 29. U.S. patent application Ser. No. 15/080,915 was a continuation in part of U.S. patent application Ser. No. 14/526,600 filed on 2014 Oct. 29. U.S. patent application Ser. No. 14/526,600 claimed the priority benefit of U.S. provisional patent application No. 61/897,245 filed on 2013 Oct. 30. U.S. patent application Ser. No. 14/526,600 was a continuation in part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application No. 61/126,047 filed on 2008 May 1. U.S. patent application Ser. No. 12/989,048 claimed the priority benefit of U.S. provisional patent application No. 61/126,027 filed on 2008 May 1.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to intrasacular devices for occluding cerebral aneurysms.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### REVIEW OF THE RELEVANT ART

There is considerable relevant art with respect to aneurysm occlusion devices. For example, U.S. patent applications 20160022445 (Ruvalcaba et al., Jan. 28, 2016, “Occlusive Device”) and 20190343664 (Ruvalcaba et al., Nov. 14, 2019, “Occlusive Device”) disclose an intrasacular aneurysm occlusion device which is made from a single continuous piece of material that is shape set into a plurality of distinct structural components, including an atraumatic tip. Also, U.S. patent application 20170156734 (Griffin, Jun. 8, 2017, “Occlusion Device”) and U.S. Pat. No. 10,285,711 (Griffin, May 14, 2019, “Occlusion Device”) discloses an intrasacular aneurysm occlusion device with a single continuous piece of material comprising axial mesh carriages configured end to end, wherein each end of each carriage is a pinch point in the continuous mesh structure. U.S. Pat. No. 10,130,372 (Griffin, Nov. 20, 2018, “Occlusion Device”), and U.S. patent applications 20150313605 (Griffin, Nov. 5, 2015, “Occlusion Device”), 20190053810 (Griffin, Feb. 21, 2019, “Occlusion Device”), 20190059909 (Griffin, Feb. 28, 2019, “Occlusion Device”) disclose an occlusive mesh with a circumferential fold line. U.S. patent application 20190269414 (Griffin, Sep. 5, 2019, “Occlusion Device”) discloses an intrasaccular occlusion device with a plurality of coaxial expandable carriages.

Intrasaccular ribbons are innovative relative to conventional coiling, but can have limitations. For example, due to the somewhat random turning and winding of a ribbon within an aneurysm sac, it can be challenging for a user of a ribbon device to ensure that a wide portion of the ribbon actually winds up being across the aneurysm neck. Also, some ribbons tend to form a pre-determined (e.g. spherical) shape which does not conform to the sac contours of an irregularly-shaped aneurysm.

U.S. Pat. No. 8,974,512 (Aboytes et al., Mar. 10, 2015, “Devices and Methods for the Treatment of Vascular Defects”), U.S. Pat. No. 8,998,947 (Aboytes et al., Apr. 7, 2015, “Devices and Methods for the Treatment of Vascular Defects”), and U.S. Pat. No. 9,844,382 (Aboytes et al., Dec. 19, 2017, “Devices and Methods for the Treatment of Vascular Defects”), and U.S. patent applications 20120239074 (Aboytes et al., Sep. 20, 2012, “Devices and Methods for the Treatment of Vascular Defects”), 20130116722 (Aboytes et al., May 9, 2013, “Devices and Methods for the Treatment of Vascular Defects”), 20150209050 (Aboytes et al., Jul. 30, 2015, “Devices and Methods for the Treatment of Vascular Defects”), 20150272590 (Aboytes et al., Oct. 1, 2015, “Devices and Methods for the Treatment of Vascular Defects”), 20150342613 (Aboytes et al., Dec. 3, 2015, “Devices and Methods for the Treatment of Vascular Defects”), 20160262766 (Aboytes et al., Sep. 15, 2016, “Devices and Methods for the Treatment of Vascular Defects”), 20180125501 (Aboytes et al., May 10, 2018, “Devices and Methods for the Treatment of Vascular Defects”), 20180132859 (Aboytes et al., May 17, 2018, “Devices and Methods for the Treatment of Vascular Defects”), 20180132862 (Aboytes et al., May 17, 2018, “Devices and Methods for the Treatment of Vascular Defects”), 20190105054 (Aboytes et al., Apr. 11, 2019, “Devices and Methods for the Treatment of Vascular Defects”), 20190105056 (Aboytes et al., Apr. 11, 2019, “Devices and Methods for the Treatment of Vascular Defects”), 20180036012 (Aboytes et al., Feb. 8, 2018, “Devices, Systems, and Methods for the Treatment of Vascular Defects”), 20190059907 (Rosqueta et al., Feb. 28, 2019, “Devices, Systems, and Methods for the Treatment of Vascular Defects”), and 20170281194 (Divino et al., Oct. 5, 2017, “Embolic Medical Devices”) disclose intrasaccular ribbons for aneurysm occlusion.

U.S. Pat. No. 10,433,853 (Divino et al., Oct. 8, 2019, “Embolic Medical Devices”) discloses an intrasaccular ribbon for aneurysm occlusion with a pre-insertion rolled configuration. U.S. patent application 20190298387 (Qin et al., Oct. 3, 2019, “Aneurysm Treatment Coils”) discloses an occlusion device with open loops and closed loops to provide balanced stiffness and flexibility. U.S. patent application 20170252190 (Becking et al., Sep. 7, 2017, “Braid Implant Delivery Systems”) discloses neurovascular devices with low profile compressibility. U.S. patent application 20150196744 (Aboytes, Jul. 16, 2015, “Devices and Method for Vascular Recanalization”) and U.S. Pat. No. 9,931,495 (Aboytes, Apr. 3, 2018, “Devices and Methods for Vascular Recanalization”) disclose a device for restoring blood flow through an obstructed blood vessel.

U.S. patent application 20190254691 (Martin et al., Aug. 22, 2019, “Flexible Intravascular Treatment Devices and Associated Systems and Methods of Use”) discloses stents with a plurality of cells and a plurality of joints between adjacent cells. U.S. patent application 20190133795 (Choubey, May 9, 2019, “Meshes, Devices and Methods for Treating Vascular Defects”) discloses stents with a plurality of strut regions and a plurality of bridge regions. U.S. patent application 20190133794 (Kusleika, May 9, 2019, “Methods and Systems for Increasing a Density of a Region of a Vascular Device”) discloses a stent with elastic members and differential porosity. U.S. Pat. No. 10,314,593 (Bardsley et al., Jun. 11, 2019, “Occlusive Devices”) discloses dual-layer inverted meshes for vascular occlusion. U.S. Pat. No. 10,327,781 (Divino et al., Jun. 25, 2019, “Occlusive Devices”) and U.S. patent application 20140135812 (Divino et al., May 15, 2014, “Occlusive Devices”) disclose intrasaccular occlusion which are filled with liquid embolic material and expand to a pre-set shape.

U.S. patent applications 20170079661 (Bardsley et al., Mar. 23, 2017, “Occlusive Devices”) and 20190269411 (Bardsley et al., Sep. 5, 2019, “Occlusive Devices”) disclose dual-layer inverted meshes for vascular occlusion. U.S. patent applications 20190282242 (Divino et al., Sep. 19, 2019, “Occlusive Devices”) and 20190290286 (Divino et al., Sep. 26, 2019, “Occlusive Devices”) disclose intrasaccular occlusion devices which are filled with liquid embolic material and expand to a pre-set shape. U.S. patent applications 20190269534 (Choubey, Sep. 5, 2019, “Thin Film Mesh Hybrid for Treating Vascular Defects”) and 20170273692 (Choubey, Sep. 28, 2017, “Thin Wall Constructions for Vascular Flow Diversion”) disclose stents with strut regions extending circumferentially about the expandable device.

U.S. Pat. No. 9,393,022 (Becking et al., Jul. 19, 2016, “Two-Stage Deployment Aneurysm Embolization Devices”) discloses embolic implants which are deployed in two stages. U.S. Pat. No. 9,157,174 (Kusleika, Oct. 13, 2015, “Vascular Device for Aneurysm Treatment and Providing Blood Flow into a Perforator Vessel”) and U.S. Pat. No. 9,561,122 (Kusleika, Feb. 7, 2017, “Vascular Device for Aneurysm Treatment and Providing Blood Flow into a Perforator Vessel”) disclose occlusion devices with heat-set strands. U.S. patent applications 20190239895 (Dawson et al., Aug. 8, 2019, “Vascular Expandable Devices”) and 20190240049 (Dawson et al., Aug. 8, 2019, “Vascular Expandable Devices”) disclose a device with a generally tubular sidewall formed by braided strands. U.S. patent application 20190192322 (Choubey et al., Jun. 27, 2019, “Vascular Flow Diversion”) discloses a device with a plurality of connector sections extending circumferentially about the device. U.S. patent application 20190069900 (Cam et al., Mar. 7, 2019, “Vascular Remodeling Device”) discloses a vascular remodeling device with a first section and a protruding section.

U.S. patent applications 20140330299 (Rosenbluth et al., Nov. 6, 2014, “Embolic Occlusion Device and Method”), 20130245667 (Marchand et al., Sep. 19, 2013, “Filamentary Devices and Treatment of Vascular Defects”), 20180206849 (Hewitt et al., Jul. 26, 2018, “Filamentary Devices for the Treatment of Vascular Defects”), 20170095254 (Hewitt et al., Apr. 6, 2017, “Filamentary Devices for Treatment of Vascular Defects”), 20180000489 (Marchand et al., Jan. 4, 2018, “Filamentary Devices for Treatment of Vascular Defects”), and 20140052233 (Cox et al., Feb. 20, 2014, “Methods and Devices for Treatment of Vascular Defects”) disclose a self-expanding globular shell which is inserted into an aneurysm sac.

U.S. patent application 20180303486 (Rosenbluth et al., Oct. 25, 2018, “Embolic Occlusion Device and Method”) discloses a self-expanding globular shell which is inserted into an aneurysm sac plus a coil which extends out from the distal end of the shell. U.S. patent application 20190223881 (Hewitt et al., Jul. 25, 2019, “Devices for Therapeutic Vascular Procedures”) discloses a self-expanding globular shell which is inserted into an aneurysm sac, wherein some shell filaments extend beyond the distal end of the shell. U.S. Pat. No. 9,955,976 (Hewitt et al., May 1, 2018, “Filamentary Devices for Treatment of Vascular Defects”) discloses a self-expanding intrasaccular globular shell with areas with different size pores. U.S. patent application 20160249934 (Hewitt et al., Sep. 1, 2016, “Filamentary Devices for Treatment of Vascular Defects”) discloses occlusive meshes with variable mesh density.

U.S. patent application 20160045201 (Rosenbluth et al., Feb. 18, 2016, “Blood Flow Disruption Devices and Methods for the Treatment of Vascular Defects”) discloses a blood flow disruption device with a porous inner flow disruption element and a porous outer flow disruption element which coaxially surrounds the inner flow disruption element. U.S. patent application 20190046209 (Plaza et al., Feb. 14, 2019, “Delivery and Detachment Systems and Methods for Vascular Implants”) discloses a system for delivering an implant device to a vascular site. U.S. patent application 20160249935 (Hewitt et al., Sep. 1, 2016, “Devices for Therapeutic Vascular Procedures”) discloses an expandable cylindrical structure made of wires with a self-expanding permeable shell at the distal end of the cylindrical structure. U.S. patent application 20160367260 (Hewitt et al., Dec. 22, 2016, “Devices for Therapeutic Vascular Procedures”) discloses an expandable cylindrical structure made of wires and a self-expanding permeable shell at the distal end of the cylindrical structure. U.S. patent application 20170128077 (Hewitt et al., May 11, 2017, “Devices for Therapeutic Vascular Procedures”) discloses methods and devices for removing a thrombus.

U.S. patent application 20120283768 (Cox et al., Nov. 8, 2012, “Method and Apparatus for the Treatment of Large and Giant Vascular Defects”) discloses a plurality of self-expanding globular shells which are inserted into an aneurysm sac. U.S. patent application 20170245862 (Cox et al., Aug. 31, 2017, “Methods and Devices for Treatment of Vascular Defects”) discloses a method for inserting a self-expanding globular shell into an aneurysm sac. U.S. patent application 20160249937 (Marchand et al., Sep. 1, 2016, “Multiple Layer Filamentary Devices for Treatment of Vascular Defects”) discloses a self-expanding multi-layer shell which is inserted into an aneurysm sac. U.S. patent application 20180271540 (Merritt et al., Sep. 27, 2018, “Systems and Methods for Embolization of Body Structures”) discloses a self-expanding shell with lobes which is inserted into an aneurysm sac. U.S. patent application 20170258473 (Plaza et al., Sep. 14, 2017, “Systems and Methods for Delivery of Stents and Stent-Like Devices”) discloses a self-expanding tubular structure which is inserted into the parent vessel of an aneurysm. U.S. Pat. No. 10,398,441 (Warner et al., Sep. 3, 2019, “Vascular Occlusion”) discloses an aneurysm occlusion system which includes a containment bag, a pusher, and a stopper ring. U.S. patent application 20190269533 (Vong et al., Sep. 5, 2019, “Stent and Stent Delivery Device”) discloses a stent made from a single woven nitinol wire.

U.S. Pat. No. 6,350,270 (Roue, Feb. 26, 2002, “Aneurysm Liner”) discloses an aneurysm liner with an extender inside the liner. U.S. Pat. No. 7,153,323 (Teoh et al., Dec. 26, 2006, “Aneurysm Liner with Multi-Segment Extender”) discloses an aneurysm liner with extender segments inside the liner. U.S. patent application 20170354402 (Lee et al., Dec. 14, 2017, “Braided Medical Devices”) discloses a vaso-occlusive member with helically-wound filaments. U.S. patent application 20190262119 (Gupta et al., Aug. 29, 2019, “Delivery Device for Use with an Embolic Material”) discloses an embolic material delivery assembly with an outer member having a lumen extending therein, a distal end region, and an inner member disposed within the lumen of the outer member. U.S. Pat. No. 5,935,148 (Villar et al., Aug. 10, 1999, “Detachable, Varying Flexibility, Aneurysm Neck Bridge”) and U.S. Pat. No. 6,063,104 (Villar et al., May 16, 2000, “Detachable, Varying Flexibility, Aneurysm Neck Bridge”) disclose an aneurysm neck bridge with varying flexibility.

U.S. patent application 20180070955 (Greene et al., Mar. 15, 2018, “Embolic Containment”) discloses systems to deliver liquid embolic material into an aneurysm. U.S. patent application 20190046210 (Bowman, Feb. 14, 2019, “Embolic Device with Shaped Wire”) discloses using a helical carrier to occlude an aneurysm. U.S. patent application 20190150932 (Cruise et al., May 23, 2019, “Embolization Device Constructed from Expansile Polymer”) discloses expandable polymer devices for aneurysm occlusion. U.S. Pat. No. 7,695,488 (Berenstein et al., Apr. 13, 2010, “Expandable Body Cavity Liner Device”) discloses an aneurysm liner with areas with different elasticities. U.S. patent application 20030028209 (Teoh et al., Feb. 6, 2003, “Expandable Body Cavity Liner Device”) discloses an aneurysm liner for treating aneurysms of various shapes and sizes. U.S. patent application 20040098027 (Teoh et al., May 20, 2004, “Expandable Body Cavity Liner Device”) discloses various aneurysm treatment devices ranging from ball stents to permeable liners.

U.S. patent applications 20060155323 (Porter et al., Jul. 13, 2006, “Intra-Aneurysm Devices”) and 20190298379 (Porter et al., Oct. 3, 2019, “Intra-Aneurysm Devices”) disclose an aneurysm occlusion device with an upper member in the dome and a lower member in the aneurysm neck. U.S. Pat. No. 9,592,363 (Griffin et al., Mar. 14, 2017, “Medical Device”) discloses a device with a shaft having an elongated inner member and an elongated tubular reinforcing member disposed over at least a portion of the inner member. U.S. patent application 20160213380 (O'Brien, et al., Jul. 28, 2016, “Occlusion Device Having Spherical Secondary Shape and Mandrel for Forming Same”) discloses a sphere made from helical memory wire. U.S. patent application 20190274691 (Sepetka et al., 9/12/2019, “Occlusive Device”) discloses a tubular braid that folds inward on itself for aneurysm occlusion.

U.S. patent applications 20190216468 (Larsen et al., Jul. 18, 2019, “Occlusive Medical Device”), 20190247053 (Inouye, Aug. 15, 2019, “Occlusive Medical Device”), 20190223883 (Anderson et al., Jul. 25, 2019, “Occlusive Medical Device with Delivery System”), 20190298380 (Inouye et al., Oct. 3, 2019, “Occlusive Medical Device with Fixation Members”), and 20190083075 (Onushko et al., Mar. 21, 2019, “Occlusive Medical Device with Sealing Member”) disclose a neck bridge to occlude a heart appendage.

U.S. Pat. No. 5,690,666 (Berenstein et al., Nov. 25, 1997, “Ultrasoft Embolism Coils and Process for Using Them”) discloses ultrasoft embolism coils. U.S. patent application 20190240050 (Dawson et al., Aug. 8, 2019, “Vascular Expandable Devices”) discloses a tubular structure made with a plurality of braided metallic elements. U.S. patent applications 20180263629 (Murphy et al., Sep. 20, 2018, “Vaso-Occlusive Device and Delivery Assembly”) and 20190254676 (Murphy et al., Aug. 22, 2019, “Vaso-Occlusive Device and Delivery Assembly”) disclose a vaso-occlusive treatment system with multi-layer wires. U.S. Pat. No. 10,426,486 (Guo et al., Oct. 1, 2019, “Vaso-Occlusive Device Delivery System”) and U.S. patent application 20170354418 (Teoh et al., Dec. 14, 2017, “Vaso-Occlusive Device Delivery System”) disclose a vaso-occlusive device delivery system with a heat-activated pusher.

U.S. patent applications 20170086851 (Wallace et al., Mar. 30, 2017, “Vaso-Occlusive Devices and Methods of Use”) and 20190201000 (Wallace et al., Jul. 4, 2019, “Vaso-Occlusive Devices”) disclose a vaso-occlusive delivery system with a pusher. U.S. Pat. No. 10,383,635 (Wallace et al., Aug. 20, 2019, “Vaso-Occlusive Devices and Methods of Use”) and U.S. patent application 20180250013 (Wallace et al., Sep. 6, 2018, “Vaso-Occlusive Devices Including a Friction Element”) disclose a vaso-occlusive system with a pusher to deliver soft embolic members. U.S. patent application 20190167270 (Chen, Jun. 6, 2019, “Vaso-Occlusive Devices with In-Situ Stiffening”) discloses a vaso-occlusive device that is constructed out of dissimilar metallic materials which cause galvanic corrosion.

U.S. patent application 20190209178 (Richter et al., Jul. 11, 2019, “Aneurysm Closure Device”) discloses occlusion of an aneurysm neck using a device with a plurality of self-expanding arms. U.S. patent application 20030212419 (West, Nov. 13, 2003, “Aneurysm Embolization Device and Deployment System”) discloses an aneurysm embolization device with a headpiece and a plurality of spherical members. U.S. patent application 20080281350 (Sepetka et al., Nov. 13, 2008, “Aneurysm Occlusion Devices”) discloses an (hourglass-shaped) occlusive device with a biocompatible matrix. U.S. patent application 20060116709 (Sepetka et al., Jun. 1, 2006, “Aneurysm Treatment Devices and Methods”) discloses a device which expands within an aneurysm sac.

U.S. patent application 20190216467 (Goyal, Jul. 18, 2019, “Apparatus and Methods for Intravascular Treatment of Aneurysms”) discloses an aneurysm neck bridge deployed in the parent vessel of the aneurysm. U.S. Pat. No. 6,346,117 (Greenhalgh, Feb. 12, 2002, “Bag for Use in the Intravascular Treatment of Saccular Aneurysms”) and U.S. Pat. No. 6,391,037 (Greenhalgh, May 21, 2002, “Bag for Use in the Intravascular Treatment of Saccular Aneurysms”) disclose a plurality of resilient filamentary members braided into a tubular sleeve with an opening to receive a clotting medium such as a platinum wire. U.S. patent application 20090287294 (Rosqueta et al., Nov. 19, 2009, “Braid-Ball Embolic Devices”) discloses “Goodness, Gracious, Great balls of wire!”

U.S. Pat. No. 4,341,218 (U, Sep. 27, 1982, “Detachable Balloon Catheter”) discloses a balloon with a hollow cylinder fastened at the neck of the balloon. U.S. Pat. No. 4,364,392 (Strother et al., Dec. 21, 1982, “Detachable Balloon Catheter”) discloses a balloon into which a carrier liquid is pumped. U.S. Pat. No. 6,511,468 (Cragg et al., Jan. 28, 2003, “Device and Method for Controlling Injection of Liquid Embolic Composition”) discloses a system to deliver liquid embolic material into an aneurysm. U.S. patent application 20190262123 (Mangiardi, Aug. 29, 2019, “Device and Method for Management of Aneurism, Perforation and Other Vascular Abnormalities”) discloses a method for treating perforations, fistulas, ruptures, dehiscence and aneurysms. U.S. patent application 20060052816 (Bates et al., Mar. 9, 2006, “Device for Treating an Aneurysm”) discloses a patch that covers an aneurysm neck.

U.S. Pat. No. 6,585,748 (Jeffree, Jul. 1, 2003, “Device for Treating Aneurysms”) discloses a permeable intrasaccular bag into which embolic coils are inserted. U.S. patent application 20190251866 (Babiker et al., Aug. 15, 2019, “Device Specific Finite Element Models for Simulating Endovascular Treatment”) discloses using finite element medical device models and computational fluid dynamics for aneurysm treatment. U.S. patent application 20090318948 (Linder et al., Dec. 24, 2009, “Device, System and Method for Aneurysm Embolization”) discloses dispensing embolic elements freely and randomly within an aneurysm cavity. U.S. patent application 20190201592 (Takahashi et al., Jul. 4, 2019, “Devices and Methods for Aneurysm Treatment”) discloses ways to reduce susceptibility artifacts in MRA images.

U.S. Pat. No. 8,597,320 (Sepetka et al., Dec. 3, 2013, “Devices and Methods for Treating Vascular Malformations”) discloses an occlusive device with a proximal collar and a distal collar. U.S. patent application 20020169473 (Sepetka et al., Nov. 14, 2002, “Devices and Methods for Treating Vascular Malformations”) discloses occlusive devices with a primary coil and secondary windings. U.S. Pat. No. 10,426,487 (Bachman et al., Oct. 1, 2019, “Devices, Systems and Methods for Enclosing an Anatomical Opening”) discloses a device with a distal-facing portion which occludes an aneurysm and a proximal-facing portion which arches over lumina of an artery. U.S. patent application 20150216684 (Enzmann et al., Aug. 6, 2015, “Dual Rotational Stent Apparatus and Method for Endovascular Treatment of Aneurysms”) discloses a coaxial stent system for aneurysm treatment.

U.S. Pat. No. 6,530,934 (Jacobsen et al., Mar. 11, 2003, “Embolic Device Composed of a Linear Sequence of Miniature Beads”) discloses an embolic device comprising a sequence of flexibly interconnected miniature beads. U.S. patent application 20190307546 (Aguilar et al., Oct. 10, 2019, “Embolic Device with Improved Neck Coverage”) discloses a helical intrasaccular device. U.S. patent application 20040010263 (Boucher et al., Jan. 15, 2004, “Expandable Preformed Structures for Deployment in Interior Body Regions”) discloses using a stylet to straighten an expandable structure during deployment into an interior body region. U.S. Pat. No. 10,405,966 (Johnson, Sep. 10, 2019, “Implantable Intraluminal Device”) discloses intraluminal stent graft devices whose walls include compliant channels which allow for fluid communication.

U.S. patent application 20190167272 (Stephens et al., Jul. 6, 2019, “Inflatable Implant”) discloses an implant with a low profile when introduced into the body and a larger profile when it is inflated with one or more filler materials. U.S. Pat. No. 10,420,862 (Sharma et al., Sep. 24, 2019, “In-Situ Forming Foams for Treatment of Aneurysms”) and U.S. patent application 20120265287 (Sharma et al., Oct. 18, 2012, “In-Situ Forming Foams for Treatment of Aneurysms”) disclose the use of in-situ forming polymer foams to treat aneurysms. U.S. patent application 20190307460 (Ferrera et al., Oct. 10, 2019, “Intrasacular Occlusion Devices Methods Processes and Systems”) discloses flexible aneurysm embolization devices made from laser cut nitinol. U.S. patent application 20090112249 (Miles et al., Apr. 30, 2009, “Medical Device for Modification of Left Atrial Appendage and Related Systems and Methods”) discloses collapsible and self-expanding devices to modify a left atrial appendage.

U.S. patent application 20190209181 (Mayer et al., Jul. 11, 2019, “Medical Device for Treating Vascular Malformations”) discloses a helical device with a coilable section and a docking section. U.S. patent application 20050142163 (Hunter et al., Jun. 30, 2005, “Medical Implants and Fibrosis-Inducing Agents”) discloses implants with fibrosis-inducing agents. U.S. patent application 20110184451 (Sahl, Jul. 28, 2011, “Membrane Implant for Treatment of Cerebral Artery Aneurysms”) discloses a cylindrical biocompatible plastic membrane used in combination with a stent. U.S. Pat. No. 7,083,643 (Whalen et al., Aug. 1, 2006, “Methods for Treating Aneurysms”) discloses filling an aneurysm sac with a fluid composition which solidifies in situ.

U.S. patent applications 20190209146 (Hebert et al., Jul. 11, 2019, “Micrograft for the Treatment of Intracranial Aneurysms and Method for Use”), 20190231328 (Hebert et al., Aug. 1, 2019, “Micrograft for the Treatment of Intracranial Aneurysms and Method for Use”), and 20190261967 (Hebert et al., Aug. 29, 2019, “Micrograft for the Treatment of Intracranial Aneurysms and Method for Use”) disclose a micrograft with a series of peaks and valleys formed by crimping. U.S. Pat. No. 10,406,010 (Bourang, Sep. 10, 2019, “Multi-Stent and Multi-Balloon Apparatus for Treating Bifurcations and Methods of Use”) discloses using two catheters and three stents to treat a bifurcated vessel. U.S. patent application 20190262002 (Benjamin, Aug. 29, 2019, “Novel Enhanced Orb-Like Intrasacular Device”) discloses an orb-shaped device with zones of flexure and open cells.

U.S. patent application 20150005807 (Lagodzki et al., Jan. 1, 2015, “Occlusion Device Including Bundle of Occlusion Wires Having Preformed Shapes”) discloses an occlusion device with shape memory wires which expand to a preformed shape. U.S. Pat. No. 10,342,548 (Duncan, Jul. 9, 2019, “Occlusion Devices and Methods of Their Manufacture and Use”) discloses a device with a lateral fringe on membranous material. U.S. Pat. No. 10,383,749 (Zhou et al., Aug. 20, 2019, “Stent and Method of Inserting a Stent into a Delivery Catheter”) discloses a stent which is radially contractable from a fully radially expanded state to a radially contracted state via elongation of the frame. U.S. patent application 20110196413 (Wallace et al., Aug. 11, 2011, “System and Method for Retaining Vaso-Occlusive Devices within an Aneurysm”) discloses an occlusive mesh made from a shape-memory alloy. U.S. patent application 20190223880 (Gerberding et al., Jul. 25, 2019, “Systems and Methods for Supporting or Occluding a Physiological Opening or Cavity”) discloses a device with a distal-facing portion which occludes an aneurysm and a proximal-facing portion which arches over lumina of an artery. U.S. Pat. No. 5,334,210 (Gianturco, Aug. 2, 1994, “Vascular Occlusion Assembly”) discloses an occlusion bag with an expanded diamond shape and an elongated flexible filler member.

U.S. patent application 20060167494 (Suddaby, Jul. 27, 2006, “Aneurysm Repair Method and Apparatus”) discloses disks pressing against inner and outer sides of an aneurysm neck. U.S. patent application 20020026217 (Baker et al., Feb. 28, 2002, “Apparatus and Method for Repair of Perigraft Flow”) discloses a device for causing thrombus between a graft and an aneurysm wall. U.S. patent application 20010034531 (Ho et al., Oct. 25, 2001, “Bioactive Three Loop Coil”) discloses an occlusion subassembly comprising a base section and a lateral protrusion fixedly attached to the base section. U.S. Pat. No. 6,855,153 (Saadat, Feb. 15, 2005, “Embolic Balloon”) and U.S. patent application 20020165572 (Saadat, Nov. 7, 2002, “Embolic Balloon”) disclose an embolic balloon which aspirates blood while expanding.

U.S. patent application 20020026210 (Abdel-Gawwad, Feb. 28, 2002, “Endovascular Aneurysm Treatment Device and Method”) discloses using an intrasacular frame and suction to collapse an aneurysm. U.S. patent application 20040254625 (Stephens et al., Dec. 16, 2004, “Inflatable Implant”) discloses an implant that is inflated with filler materials. U.S. patent application 20060149309 (Paul et al., Jul. 6, 2006, “Inverting Occlusion Devices, Methods, and Systems”) discloses inverted vascular occlusion devices. U.S. Pat. No. 4,638,803 (Rand, Jan. 27, 1987, “Medical Apparatus for Inducing Scar Tissue Formation in a Body”) discloses a balloon coated with thrombosis-inducing material. U.S. Pat. No. 6,958,061 (Truckai et al., Oct. 25, 2005, “Microspheres with Sacrificial Coatings for Vaso-Occlusive Systems”) discloses using a fluid to deliver microspheres for vascular occlusion. U.S. Pat. No. 5,041,090 (Scheglov et al., Aug. 20, 1991, “Occluding Device”) discloses using nested balloons for occlusion.

### SUMMARY OF THE INVENTION

This invention is a “bowl-of-spaghetti” type intrasacular aneurysm occlusion device. This device can include: a catheter which is inserted into a person's vasculature; one or more embolic coils which are delivered through the catheter into an aneurysm sac; a neck bridge (such as a braided or woven stent, lattice, mesh, and/or framework) which self-expands into a bowl or cup shape within the aneurysm sac, expanding to a diameter which is larger than the maximum diameter of the aneurysm neck; an opening in the neck bridge through which the coils are inserted into the aneurysm sac; and a closure mechanism which closes the opening after the coils have been inserted into the aneurysm sac. It is a called a “bowl-of-spaghetti” type device because, when viewed from a side perspective, the neck bridge can look like a bowl and the embolic coils can look like spaghetti noodles in the bowl.

In an example, a neck bridge can self expand within the aneurysm sac. In an example, a neck bridge can expand into an upward-opening-convex shape, such as a bowl or cup shape. In an example, a neck bridge can be a braided or woven stent, lattice, mesh, net, and/or framework. In an example, a neck bridge can be a 3D printed stent, lattice, mesh, and/or framework. In an example, a neck bridge can be 3D printed as one, continuous piece of mesh. In an example, a neck bridge can be a multi-layer neck bridge which is made by folding and/or compressing a generally spherical or ellipsoidal stent, lattice, mesh, or framework into a multi-layer bowl or cup shaped stent, lattice, mesh, or framework. In an example, a neck bridge can be a multi-layer neck bridge which is made by folding and/or compressing a generally spherical or ellipsoidal stent, lattice, mesh, or framework within the aneurysm sac. In an example, a neck bridge can be a multi-layer neck bridge which is made by folding, twisting, and/or inverting a generally tubular stent, lattice, mesh, or framework into a multi-layer bowl or cup shaped stent, lattice, mesh, or framework.

In an example, a neck bridge can expand to a diameter which is at least 10% larger than the maximum diameter of the aneurysm neck. In an example, a neck bridge can expand to a diameter which is at least 85% of the maximum diameter of the aneurysm sac. In an example, a neck bridge can expand to a diameter which is at least 10% larger than the maximum diameter of the aneurysm neck and at least 85% of the maximum diameter of the aneurysm sac. In an example, a neck bridge can expand to a diameter which is at least 25% larger than the maximum diameter of the aneurysm neck and at least 95% of the maximum diameter of the aneurysm sac.

In an example, a catheter can connect to and/or protrude into the opening in a neck bridge. In an example, a closure mechanism can be remotely-activated by a device user via the application of electromagnetic energy. In an example, a closure mechanism can close the opening in a neck bridge and also detach an embolic coil which has been inserted into the aneurysm sac. In an example, a closure mechanism can further detach a catheter from a neck bridge so that the catheter can be withdrawn from the person's vasculature.

In an example, this invention can also be embodied in a method for occluding an aneurysm. This method can involve the steps of: inserting a catheter into a person's vasculature; inserting a neck bridge on (or through) the catheter into an aneurysm sac in the person's vasculature; expanding the neck bridge into a bowl or cup shape within the aneurysm sac, wherein the neck bridge is expanded to a diameter which is larger than the maximum diameter of the aneurysm neck; inserting one or more embolic coils into the aneurysm sac through the catheter and through an opening in the neck bridge; closing the opening in the neck bridge; and withdrawing the catheter from the person's vasculature.

This “bowl-of-spaghetti” type device has the potential to provide more consistent and stable coverage of an aneurysm neck than intrasacular embolic ribbons, provide higher packing density and less chance of protrusion into a parent vessel than coils alone, and provide better conformity to the walls of irregularly-shaped aneurysms than hollow braid-ball devices with pre-determined (e.g. spherical or barrel) shapes. This “bowl-of-spaghetti” type intrasacular aneurysm occlusion device has the potential to offer good, stable coverage of an aneurysm neck and high packing density of an aneurysm sac for easy-to-deploy, rapid, and thorough occlusion of a cerebral aneurysm.

### BRIEF INTRODUCTION TO THE FIGURES

FIG. 1 shows a larger-scale side-view of an intrasacular aneurysm occlusion device as an expandable proximal member (e.g. neck bridge) is being inserted into an aneurysm sac.

FIG. 2 shows a larger-scale side-view of this same intrasacular aneurysm occlusion device after the expandable proximal member (e.g. neck bridge) has been expanded and covers the aneurysm neck.

FIG. 3 shows a larger-scale side-view of this same intrasacular aneurysm occlusion device after longitudinal embolic members (e.g. coils) have been inserted into the aneurysm sac through an opening in the expandable proximal member (e.g. neck bridge).

FIG. 4 shows a smaller-scale side view of this same intrasacular aneurysm occlusion device, wherein the opening in the expandable proximal member (e.g. neck bridge) is open to allow the embolic members (e.g. coils) to be inserted into the aneurysm sac.

FIG. 5 shows a smaller-scale side view of this same intrasacular aneurysm occlusion device, wherein the opening in the expandable proximal member (e.g. neck bridge) is closed after the embolic members (e.g. coils) have been inserted into the aneurysm sac.

### DETAILED DESCRIPTION OF THE FIGURES

In an example, a “bowl-of-spaghetti” type intrasacular aneurysm occlusion device can comprise: a longitudinal lumen (e.g. a catheter) which is configured into be inserted into a person's vasculature; one or more longitudinal embolic members (e.g. coils) which are configured to be delivered through the longitudinal lumen into an aneurysm sac in the person's vasculature; an expandable proximal member (e.g. a bowl-shaped neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck; an opening in the expandable proximal member through which the longitudinal embolic members are inserted into the aneurysm sac; and a closure mechanism which closes the opening after the longitudinal embolic members have been inserted into the aneurysm sac. This is a called “bowl-of-spaghetti” device because, from a lateral perspective, the expandable proximal member looks like a bowl and the plurality of longitudinal embolic members look like spaghetti noodles in the bowl.

In an example, a “bowl-of-spaghetti” type intrasacular aneurysm occlusion device can also be described as comprising: a catheter which is inserted into a person's vasculature; coils which are delivered through the catheter into an aneurysm sac; a bowl-shaped neck bridge which self-expands within a proximal portion the aneurysm sac to a diameter which is larger than the aneurysm neck; an opening in the neck bridge through which the coils are inserted into the aneurysm sac; and a closure mechanism which closes the opening after the coils have been inserted into the aneurysm sac.

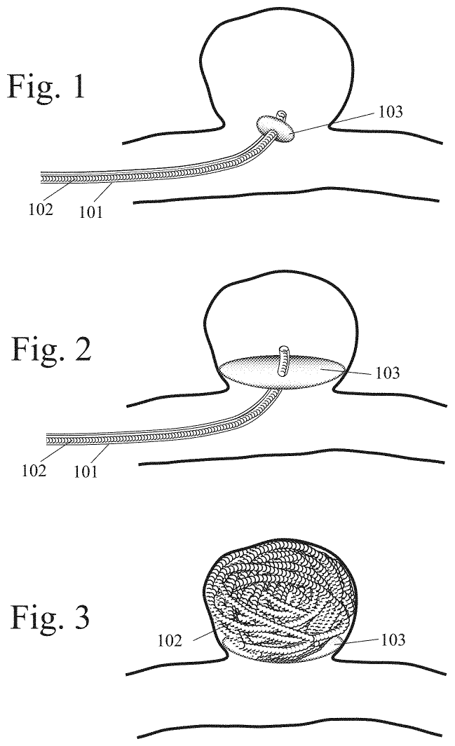

FIGS. 1 through 5 show an example of a “bowl-of-spaghetti” type intrasacular aneurysm occlusion device comprising: a longitudinal lumen (e.g. a catheter) which is configured into be inserted into a person's vasculature; one or more longitudinal embolic members (e.g. coils) which are configured to be delivered through the longitudinal lumen into an aneurysm sac in the person's vasculature; an expandable proximal member (e.g. a bowl-shaped neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck; an opening in the expandable proximal member through which the longitudinal embolic members are inserted into the aneurysm sac; and a closure mechanism which closes the opening after the longitudinal embolic members have been inserted into the aneurysm sac.

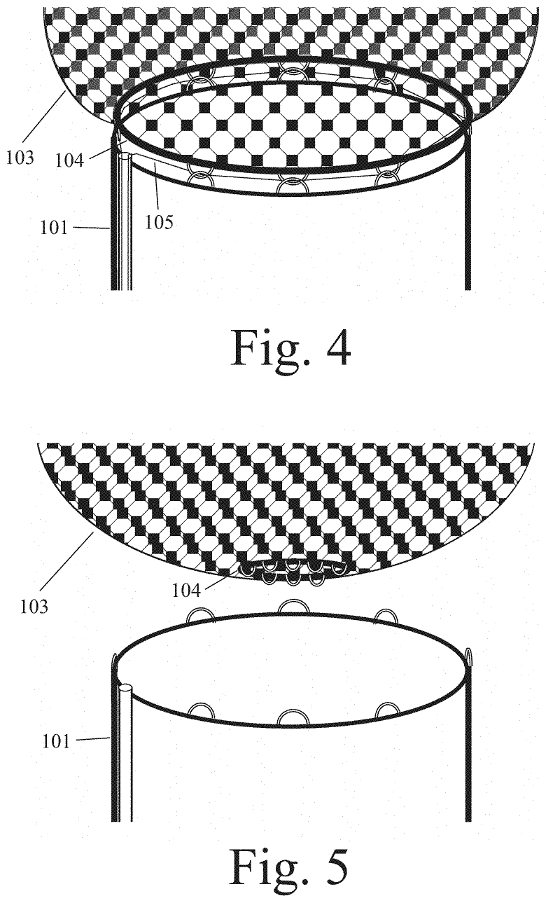

FIGS. 1 through 5 show an example of a “bowl-of-spaghetti” type intrasacular aneurysm occlusion device. FIGS. 1 through 3 show three, sequential, larger-scale, side views of this device. FIG. 1 shows this device at a first time, as the expandable proximal member is being inserted into an aneurysm sac. FIG. 2 shows this device at a second time, after the expandable proximal member has been expanded within the aneurysm sac. FIG. 3 shows this device at a third time, after longitudinal embolic members have been inserted into the aneurysm sac through a hole (e.g. opening) in the expandable proximal member. FIGS. 4 and 5 show two sequential, smaller-scale, side views of this device. FIG. 4 shows this device when the hole (e.g. opening) in the expandable proximal member is open to allow the embolic members to be inserted into the aneurysm sac. FIG. 5 shows this device when the hole (e.g. opening) in the expandable proximal member has been closed after the embolic members have been inserted into the aneurysm sac.

With respect to specific components, FIGS. 1 through 3 show a “bowl-of-spaghetti” type intrasacular aneurysm occlusion device comprising: longitudinal lumen 101 (e.g. a catheter) which is configured into be inserted into a person's vasculature; one or more longitudinal embolic members 102 (e.g. coils) which are configured to be delivered through the longitudinal lumen into an aneurysm sac in the person's vasculature; and an expandable proximal member 103 (e.g. a bowl-shaped neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck. FIGS. 4 and 5 show this device as further comprising: an opening 104 in the expandable proximal member through which the longitudinal embolic members are inserted into the aneurysm sac; and a (remote-activated) closure mechanism 105 which closes the opening after the longitudinal embolic members have been inserted into the aneurysm sac.

The example shown in FIGS. 1 through 5 can also be described as a “bowl-of-spaghetti” type intrasacular aneurysm occlusion device comprising: a catheter which is inserted into a person's vasculature; coils which are delivered through the catheter into an aneurysm sac; a bowl-shaped neck bridge which self-expands within a proximal portion the aneurysm sac to a diameter which is larger than the aneurysm neck; an opening in the neck bridge through which the coils are inserted into the aneurysm sac; and a closure mechanism which closes the opening after the coils have been inserted into the aneurysm sac. Example variations and components disclosed elsewhere in this application or in priority-linked applications can also be applied, where relevant, to the example shown here in FIGS. 1 through 5.

In this example, the longitudinal lumen is configured to be inserted into a blood vessel, wherein this blood vessel is the parent vessel from which an aneurysm has formed. The longitudinal embolic members are configured to travel through the longitudinal lumen and be inserted into the aneurysm sac. The expandable proximal member is configured to travel through the longitudinal lumen, be inserted into the aneurysm sac, and self expand within the aneurysm sac. The expandable proximal member is configured to substantially occlude the aneurysm neck after it is expanded. The longitudinal embolic members are inserted into the aneurysm sac through a central opening of the expandable proximal member. The expandable proximal member prevents the longitudinal flexible members from protruding into the parent vessel of the aneurysm. In this example, the expandable proximal member is a mesh. In this example, the closure mechanism is a cord. In this example, pulling a cord detaches loops which hold an elastic ring in the expandable proximal member open, thereby closing the opening in the proximal expandable member.

In an example, a longitudinal lumen can be a removable catheter. In an example, a longitudinal lumen can be configured to be inserted into a blood vessel from which an aneurysm sac has formed. In an example, one or more longitudinal embolic members can be embolic coils. In an example, a longitudinal embolic member can be configured to travel through a longitudinal lumen and inserted into an aneurysm sac. In an example, the function of this device can be described as aneurysm coil jailing using an expandable proximal member. The expandable proximal member prevents the one or more longitudinal flexible members (e.g. coils) from protruding into the parent vessel of an aneurysm. In an example, an expandable proximal member can be an expandable intrasacular mesh, braid, lattice, or framework. In an example, an expandable proximal member can be toroidal. In an example, an expandable proximal member can be an expandable (toroidal) proximal member that is configured to be inserted into and expanded within aneurysm sac. In an example, an expandable (toroidal) proximal member can remain in the aneurysm sac after implantation of the device.

In an example, an expandable proximal member can self expand after it is inserted into an aneurysm sac. In an example, an expandable (toroidal) proximal member can self expand because it comprises shape memory material. In an example, an expandable proximal member can comprise a mesh, network, lattice, or radial array of wires or other stiff fibers. In an example, an expandable proximal member can be reinforced with wires or other stiff fibers. In an example, an expandable proximal member can be made from metal, a polymer, or both. In an example, an expandable proximal member can be made with nitinol (or other nickel titanium alloy), cobalt-chrome alloy (cobalt chromium), gold, palladium, platinum, steel (e.g. stainless steel), tantalum, titanium, and/or tungsten.

In an example, an expandable proximal member can expand in a radial-expansion plane that is substantively parallel with the plane defined by a central circumference of an aneurysm neck. In an example, an expandable proximal member can be expanded at a distance from a central circumference of an aneurysm neck from which its radial expansion contacts the aneurysm sac at the sac's maximum circumference. In an example, an expandable proximal member can be expanded at a distance from a central circumference of the aneurysm neck from which its radial expansion contacts the aneurysm sac at a sac circumference that is greater than the central circumference of the aneurysm neck. In an example, an expandable proximal member can function as an intrasacular stent or neck bridge.

In an example, an expandable proximal member can have a toroidal shape, bowl shape, convex lens shape, cup shape, paraboloid (of revolution) shape, inverted umbrella shape, or hemispherical shape. In an example, an expandable proximal member can have an annular shape, cylindrical shape doughnut shape, ring shape, tire shape, or wheel shape. In an example, an expandable proximal member can have a disk shape. In an example, an expandable proximal member can have a dome shape. In an example, an expandable proximal member can be a spherical stent which has been collapsed into a hemispherical shape. In an example, an expandable proximal member can comprise a spherical stent which is collapsed into a hemisphere in order to create a double-layer barrier near an aneurysm neck.

In an example, an expandable proximal member can have an adjustable opening through which one or more embolic members are inserted into an aneurysm sac. In an example, one or more longitudinal embolic members can be inserted into an aneurysm sac through an opening in a expandable proximal member after the expandable proximal member has been expanded within an aneurysm sac. In an example, a plurality of embolic members can be inserted into an aneurysm sac through a central opening in an expandable proximal member and the expandable proximal member can have a sufficiently tight fit with the aneurysm walls that none of the embolic members escape into the parent blood vessel. In an example, embolic members can be inserted into an aneurysm sac through a one-way valve in a hole (e.g. opening) in an expandable proximal member.

In an example, an expandable proximal member can further comprise an opening through which embolic members are introduced into the enclosure and a closure mechanism, associated with the opening, wherein the closure mechanism is configured to be actuated to reduce a size of the opening. In an example, a hole (e.g. opening) in an expandable proximal member can be closed after an aneurysm sac has been filled with embolic members inserted through the hole. In an example, a closure mechanism can be integrated into an expandable proximal member to prevent embolic members from escaping through the opening by which they were inserted into the expandable proximal member. In an example, a closure mechanism can be remotely actuated by a device user to reduce a size of the opening. In an example, a closure mechanism can comprise a one-way valve that automatically lets embolic members into the aneurysm sac, but does not let them out. In an example, a closure mechanism can include a cord which is pulled from a remote location outside the body to detach an expandable proximal member from a catheter and close an opening in the expandable proximal member.

In an example, a closure mechanism can be selected from the group consisting of: drawstring, elastic ring, loop, and tie-off. In an example, a closure mechanism can be a pull cord or wire. In an example, a closure mechanism can be a plug or valve. In an example, a closure mechanism can be selected from the group consisting of: adhesive, electromagnetic energy, fusible member, and fusion. In an example, a closure mechanism can be selected from the group consisting of: clasp, clip, shape-memory material, and snap. In an example, a closure mechanism can be selected from the group consisting of: cap, seal, and threaded member.

In an example, a “bowl-of-spaghetti” type aneurysm occlusion device can comprise: (a) a catheter; (b) one or more embolic coils which are inserted through the catheter into an aneurysm sac; (b) an inflatable toroidal neck bridge which is expanded to a diameter which is larger than the diameter of the aneurysm neck; (e) a (central) opening in the neck bridge through which the coils are inserted into the aneurysm sac; and (f) a closure mechanism which closes the opening after the coils have been inserted into the aneurysm sac. In an alternative example, a “bowl-of-spaghetti” type aneurysm occlusion device can comprise: (a) a catheter; (b) one or more helical embolic coils which are inserted through the catheter into an aneurysm sac; (c) a braided multi-layer neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a braided neck bridge (e.g. stent, lattice, mesh, or framework) which expands (e.g. self-expands) to a bowl or cup shape within the aneurysm sac, expanding to a diameter which is at least 20% larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a braided Nitinol neck bridge (e.g. stent, framework, lattice, or mesh) which is inserted into the aneurysm sac in a first shape, then is expanded (e.g. self-expands) within the aneurysm sac into an ellipsoidal second shape, and then is compressed and/or folded into a bowl or cup third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can be embodied in: (a) a catheter; (b) one or more embolic coils which are inserted through the catheter into an aneurysm sac; (c) a braided, knitted, or woven neck bridge (e.g. stent, lattice, mesh, or framework) which expands (e.g. self-expands) within the aneurysm sac, expanding to a diameter which is at least 90% of the maximum diameter of the aneurysm sac in a plane which is parallel to the plane of the aneurysm neck; (d) an opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a double-layer neck bridge (e.g. stent, lattice, mesh, or framework) which expands (e.g. self-expands) to a hemispherical shape within an aneurysm sac, expanding to a diameter which is larger than the diameter of the aneurysm neck, wherein the neck bridge is created by compressing a spherical mesh into a double-layer hemispherical mesh; (d) a (central) opening in the neck bridge through which the coils are inserted into the aneurysm sac; and (e) a closure mechanism which closes the opening after the coils have been inserted into the aneurysm sac.

In an example, a device to occlude an aneurysm can comprise: (a) a catheter; (b) one or more embolic coils which are inserted through the catheter into an aneurysm sac; (c) a multi-layer neck bridge (e.g. stent, lattice, mesh, or framework) which expands (e.g. self-expands) to a bowl or cup shape within the aneurysm sac, expanding to a diameter which is larger than the maximum diameter of the aneurysm neck, wherein the multi-layer neck bridge is made from a tubular braid which is tied (or twisted or otherwise radially constrained) at a selected location along the length of the tube and then inverted (or folded or everted) over itself relative to that selected location; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can be embodied in: (a) a catheter; (b) one or more embolic coils which are inserted through the catheter into an aneurysm sac; (c) a multi-layer, upward-facing-convex neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) made with nitinol cobalt chromium which expands (e.g. self-expands) into a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) within an aneurysm sac, expanding into a bowl or cup shape with a diameter which is larger than the diameter of the aneurysm neck, wherein the proximal half of the neck bridge has a first porosity level, wherein the distal half of the neck bridge has a second porosity level, and wherein the second porosity level is greater than the first porosity level; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck, wherein the neck bridge further comprises at least one radiopaque marker; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac at a location from which it contacts the aneurysm sac at a sac circumference that is greater than the central circumference of the aneurysm neck; (d) a (central) opening in the neck bridge through which the coils are inserted into the aneurysm sac; and (e) a closure mechanism which closes the opening after the coils have been inserted into the aneurysm sac.

In an example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) into a hemispherical shape within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type intrasacular aneurysm occlusion device can be embodied in: (a) a catheter; (b) one or more embolic coils which are inserted through the catheter into an aneurysm sac, wherein the coils have an average first cross-sectional area; (c) a neck bridge (e.g. stent, lattice, mesh, or framework) which expands (e.g. self-expands) to a bowl or cup shape within the aneurysm sac, expanding to a diameter which is larger than the diameter of the aneurysm neck; (d) a (central) opening in the neck bridge through which the coils are inserted into the aneurysm sac, wherein the opening has a second cross-sectional area when open and a third cross-sectional area when closed, wherein the second cross-sectional area is at least 10% greater than the first cross-sectional area, and wherein the first cross-sectional area is at least 10% greater than the third cross-sectional area; and (e) a closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises an electromagnetic actuator. In another example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to an inverted umbrella shape within an aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are transported through the catheter and inserted into an aneurysm sac; (c) a neck bridge (e.g. stent, lattice, mesh, or framework) which is transported through the catheter in a radially-more-constrained first configuration, inserted into the aneurysm sac, and then expanded (e.g. self expanded) from the first configuration to a radially-less-constrained second hemispherical configuration, wherein the second configuration has a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a device to occlude an aneurysm can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a hydrogel closure mechanism which closes the opening after the coils have been inserted into the aneurysm sac. In yet another embodiment, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a tensile ring which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) an electromagnetic closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a non-central opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can comprise: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a opening in the outer (e.g. peripheral) half of the cross-sectional area neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an elastic-ring opening in the neck bridge through which the catheter protrudes and through which coils are inserted into the aneurysm sac, wherein the elastic-ring closes when the catheter is removed from the opening.

In an example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in base of the neck bridge through which the coils are inserted into the aneurysm sac; (e) an electromagnetic detachment and closure mechanism which detaches one or more segment of embolic coils and closes the opening so that the segments do not escape out of the aneurysm sac. In an alternative example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) a closure mechanism which detaches a segment of embolic coil, withdraws the catheter from the opening, and closes the opening.

A “bowl-of-spaghetti” type intrasacular aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) one or more closure mechanisms which detach one or more embolic coils, withdraw the catheter from the opening, and close the opening after the aneurysm sac has been filled with one or more embolic coils. In another example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, wherein neck of the aneurysm has a maximum diameter with a first length, wherein the aneurysm sac has a maximum diameter (in a plane which is parallel to the plane of the aneurysm neck) with a second length, wherein the neck bridge is expanded within the aneurysm sac to a third diameter with a third length, wherein the third length is greater than 120% of the first length, and wherein the third length is greater than 80% of the second length; (d) an opening in the neck bridge through which the coils are inserted into the aneurysm sac; and (e) a closure mechanism which closes the opening after the coils have been inserted into the aneurysm sac.

In one possible embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is at least 10% larger than the diameter of the aneurysm neck; (d) an opening in the neck bridge through which the coils are inserted into the aneurysm sac; and (e) a remotely-activated closure mechanism which closes the opening after the coils have been inserted into the aneurysm sac. Alternatively, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) with a helical wire which expands (e.g. self-expands) into a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a device to occlude an aneurysm can comprise: (a) a catheter; (b) one or more embolic coils which are inserted through the catheter into an aneurysm sac; (c) a Nitinol neck bridge (e.g. stent, framework, lattice, or mesh) which is inserted into the aneurysm sac in a first shape, then expanded (e.g. self-expands) within the aneurysm sac into an ellipsoidal second shape, and then compressed and/or folded into a bowl or cup third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a shaped neck bridge (e.g. stent, lattice, mesh, or framework) which is inserted into the aneurysm sac, is expanded (e.g. self-expands) into a substantially spherical shape within the aneurysm sac, and then is collapsed into a double-layer, upward-opening, bowl shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can be embodied in: (a) a catheter; (b) one or more embolic coils which are inserted through the catheter into an aneurysm sac; (c) a upwardly-concave neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a longitudinal lumen (e.g. a catheter); (b) one or more longitudinal embolic members (e.g. embolic coils) which are inserted into an aneurysm sac; (c) an expandable proximal member (e.g. a bowl or cup shaped neck bridge) which is expanded (e.g. self-expanded) within a proximal portion the aneurysm sac to a diameter which is larger than the diameter of the aneurysm neck; (d) an opening in the expandable proximal member through which the longitudinal embolic members are inserted into the aneurysm sac; and (e) a closure mechanism which closes the opening after the longitudinal embolic members have been inserted into the aneurysm sac.

In an example, a “bowl-of-spaghetti” type intrasacular aneurysm occlusion device can be embodied in: (a) a longitudinal lumen (e.g. a catheter); (b) one or more longitudinal embolic members (e.g. embolic coils) which are inserted into an aneurysm sac; (c) an expandable proximal member (e.g. an upward-opening convex stent) which is expanded (e.g. self-expanded) within a proximal portion the aneurysm sac to a diameter which is larger than the diameter of the aneurysm neck; (d) an opening in the expandable proximal member through which the longitudinal embolic members are inserted into the aneurysm sac; and (e) a closure mechanism which closes the opening after the longitudinal embolic members have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the expandable proximal member through which the coils are inserted into the aneurysm sac; (e) an electrical coil detachment (e.g. cutting) mechanism which detaches (e.g. cuts) one or more segments of embolic coils after a portion of the embolic coils has been inserted into the aneurysm sac; and (f) a closure mechanism which closes the opening after the coils have been inserted into the aneurysm sac.

In an embodiment, a device to occlude an aneurysm can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member is a spherical stent which has been collapsed or folded into a double-layer hemispherical shape; (d) a (central) opening in the neck bridge through which the coils are inserted into the aneurysm sac; and (e) a closure mechanism which closes the opening after the coils have been inserted into the aneurysm sac. In yet another embodiment, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism is a rotatable closure mechanism.

A “bowl-of-spaghetti” type aneurysm occlusion device can alternatively comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism comprises electromagnetic energy which is applied to the opening. In another example, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises expansion of a hydrogel.

In an example, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a spring or elastic member with a first configuration which does not block (or otherwise close) the opening and a second configuration which blocks (or otherwise closes) the opening, wherein the spring or elastic member is changed from its first configuration to its second configuration by stopping the transmission of electromagnetic energy.

A “bowl-of-spaghetti” type intrasacular aneurysm occlusion device can also comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism is closed by the transmission of electrical energy to it. Alternatively, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) to an inverted dome shape within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a device to occlude an aneurysm can comprise: (a) a catheter; (b) one or more embolic coils which are inserted through the catheter into an aneurysm sac; (c) an expandable proximal member (e.g. neck bridge) which expands (e.g. self-expands) within the aneurysm sac so as to completely cover the inside of the aneurysm neck; (d) a (central) opening in the neck bridge through which the coils are inserted into the aneurysm sac; and (e) a closure mechanism which reduces the size of the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a remotely-controlled (one-way) valve with a first configuration allows coils to be inserted through the opening into the aneurysm sac, but does not allow coils to escape out of the aneurysm sac, and a second configuration which allows coils to be pulled out of the aneurysm sac.

In an example, a “bowl-of-spaghetti” type aneurysm occlusion device can comprise: (a) a catheter; (b) one or more embolic coils which are inserted through the catheter into an aneurysm sac; (c) an upward-facing-convex neck bridge (e.g. stent, lattice, mesh, or framework) which expands (e.g. self-expands) within an aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In an alternative example, a “bowl-of-spaghetti” type aneurysm occlusion device can comprise: (a) a catheter; (b) one or more embolic coils which are inserted through the catheter into an aneurysm sac; (c) an upward-facing-convex neck bridge self-expands within the aneurysm sac, expanding to a diameter which is larger than the diameter of the aneurysm neck, wherein the neck bridge is made from nitinol (or other nickel titanium alloy), cobalt-chrome alloy (cobalt chromium), gold, palladium, platinum, steel (e.g. stainless steel), tantalum, titanium, and/or tungsten; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type method for occluding an aneurysm can comprise: (a) inserting a braided neck bridge (e.g. stent, lattice, mesh, or framework) into an aneurysm sac; (b) allowing the neck bridge to self-expand into a spherical or ellipsoidal first shape; (c) axially contracting, compressing, and/or folding the neck bridge from the first shape into a double-layer bowl or cup second shape; (d) inserting embolic coils into the aneurysm sac through an opening in the neck bridge; and (e) closing the opening in the neck bridge. In another example, a “bowl-of-spaghetti” type method for occluding an aneurysm can comprise: (a) inserting a neck bridge (e.g. stent, lattice, mesh, or framework) into an aneurysm sac; (b) expanding the neck bridge in the aneurysm sac into a hemispherical shape with a diameter which is wider than the diameter of the aneurysm neck; (c) inserting embolic coils into the aneurysm sac through an opening in the neck bridge; and (d) closing the opening in the neck bridge.

In an example, a “bowl-of-spaghetti” type method for occluding an aneurysm can comprise: (a) inserting a neck bridge (e.g. stent, lattice, mesh, or framework) into an aneurysm sac; (b) expanding the neck bridge in the proximal half of the aneurysm sac into a convex shape with a diameter which is wider than the diameter of the aneurysm neck; (c) inserting embolic coils into the aneurysm sac through an opening in the neck bridge; and (d) closing the opening in the neck bridge. Alternatively, a aneurysm occlusion method can be embodied in: (a) inserting a neck bridge (e.g. stent, lattice, mesh, or framework) into an aneurysm sac; (b) expanding the neck bridge into a bowl or cup shape with a diameter which is between 90% and 100% of the maximum diameter of the aneurysm sac; (c) inserting embolic coils into the aneurysm sac through an opening in the neck bridge; and (d) closing the opening in the neck bridge. In another example, a “bowl-of-spaghetti” type method for occluding an aneurysm can comprise: (a) inserting a braided neck bridge (e.g. stent, lattice, mesh, or framework) into an aneurysm sac; (b) releasing the neck bridge to self expand into a bowl or cup shape with a diameter which is wider than the diameter of the aneurysm neck; (c) inserting embolic coils into the aneurysm sac through an opening in the neck bridge; and then (d) closing the opening in the neck bridge.

In an embodiment, a “bowl-of-spaghetti” type intrasacular aneurysm occlusion device can be embodied in: (a) a catheter; (b) one or more embolic coils which are inserted through the catheter into an aneurysm sac; (c) a 3D-printed neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a device to occlude an aneurysm can comprise: (a) a catheter; (b) one or more embolic coils which are inserted through the catheter into an aneurysm sac; (c) a braided neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can comprise: (a) a catheter; (b) one or more helical embolic coils which are inserted through the catheter into an aneurysm sac; (c) a braided neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a braided, knitted, or woven neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a cross-sectionally-asymmetric neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a expandable proximal member (e.g. neck bridge) which expands (e.g. self-expands) to a disk shape within the aneurysm sac, expanding to a diameter which is larger than the diameter of the aneurysm neck; (d) a (central) opening in the neck bridge through which the coils are inserted into the aneurysm sac; (e) a coil detachment (e.g. cutting) mechanism which detaches (e.g. cuts) one or more segments of embolic coils after a portion of the embolic coils has been inserted into the aneurysm sac; and (f) a closure mechanism which closes the opening after the coils have been inserted into the aneurysm sac.

In an example, a device to occlude an aneurysm can comprise: (a) a catheter; (b) one or more embolic coils which are inserted through the catheter into an aneurysm sac; (c) a multi-layer, arcuate neck bridge (e.g. stent, lattice, mesh, or framework) which expands (e.g. self-expands) within the aneurysm sac, expanding to a diameter which is larger than the maximum diameter of the aneurysm neck; (d) a (central) opening in the neck bridge through which the coils are inserted into the aneurysm sac; and (e) an electromagnetic closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a multi-layer, upward-facing-convex neck bridge (e.g. stent, lattice, mesh, or framework) which is expanded within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac, expanding to a diameter which is larger than the maximum diameter of the aneurysm neck; (d) a (central) opening in the neck bridge through which the catheter extends and through which the coils are inserted into the aneurysm sac; and (e) a closure mechanism which retracts the catheter from the opening and closes the opening after the coils have been inserted into the aneurysm sac.

A “bowl-of-spaghetti” type intrasacular aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) within an aneurysm sac, expanding into a bowl or cup shape with a diameter which is larger than the diameter of the aneurysm neck, wherein the proximal half of the neck bridge has a first mesh density, wherein the distal half of the neck bridge has a second mesh density, and wherein the second mesh density is less than the first mesh density; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck, wherein the neck bridge further comprises a radiopaque marker at its proximal end; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type intrasacular aneurysm occlusion device can be embodied in: (a) a catheter; (b) one or more embolic coils which are inserted through the catheter into an aneurysm sac; (c) a neck bridge (e.g. stent, lattice, mesh, or framework) which self-expands into a barrel shape within the aneurysm sac, wherein we all have fun when the barrel is rolled out, with due credit to the Andrews Sisters; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self expands) to a bowl or cup shaped within the aneurysm sac, expanding to a diameter which is larger than the maximum diameter of the aneurysm neck, but smaller than the maximum diameter of the aneurysm sac; (d) a (central) opening in the neck bridge through which the coils are inserted into the aneurysm sac; and (e) a closure mechanism which closes the opening after the coils have been inserted into the aneurysm sac. In another example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within an aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In an alternative example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a MEMS actuator.

A “bowl-of-spaghetti” type aneurysm occlusion device can also comprise: (a) a catheter; (b) one or more embolic coils which are transported through the catheter and inserted into an aneurysm sac; (c) a neck bridge (e.g. stent, lattice, mesh, or framework) which is transported through the catheter in a radially-more-constrained first configuration, inserted into the aneurysm sac, and then expanded (e.g. self expanded) into a radially-less-constrained second configuration, wherein the second configuration has a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are transported through the catheter and inserted into an aneurysm sac; (c) a neck bridge (e.g. stent, lattice, mesh, or framework) which is transported through the catheter in a radially-more-constrained first configuration, inserted into the aneurysm sac, and then expanded (e.g. self expanded) from the first configuration to a radially-less-constrained second disk-shaped configuration, wherein the second configuration has a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a magnetic closure mechanism which closes the opening after the coils have been inserted into the aneurysm sac. Alternatively, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) an adhesive closure mechanism which closes the opening after the coils have been inserted into the aneurysm sac.

In an example, a “bowl-of-spaghetti” type intrasacular aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) an electromagnetic closure mechanism which closes the opening after the coils have been inserted into the aneurysm sac, wherein the closure mechanism is remotely-activated by a device user. In another example, a “bowl-of-spaghetti” type aneurysm occlusion device can comprise: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a non-central opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a plurality of non-central openings in the neck bridge through which the coils are inserted into the aneurysm sac; and (e) one or more closure mechanisms which close the plurality of openings after the coils have been inserted into the aneurysm sac. In another example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an off-center opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in base of the neck bridge through which the coils are inserted into the aneurysm sac; and (e) a closure mechanism which closes the opening after the coils have been inserted into the aneurysm sac. In an alternative example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) a closure mechanism which detaches an embolic coil, withdraws the catheter from the opening, and closes the opening.

In an example, a device to occlude an aneurysm can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter protrudes and through which the coils are inserted into the aneurysm sac, wherein the opening automatically closes when the catheter is removed from the opening. In another example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a first diameter, wherein the aneurysm neck has a second diameter, wherein the aneurysm sac has a maximum third diameter in a plane which is parallel to the aneurysm neck, and wherein the first diameter is at least 110% of the second diameter and at least 70% of the third diameter; (d) an opening in the neck bridge through which the coils are inserted into the aneurysm sac; and (e) a closure mechanism which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, wherein neck of the aneurysm has a maximum diameter with a first length, wherein the aneurysm sac as maximum diameter (in a plane which is parallel to the plane of the aneurysm neck) with a second length, wherein the neck bridge is expanded within the aneurysm sac to a third diameter with a third length, wherein the third length is greater than 110% of the first length, and wherein the third length is greater than 90% of the second length; (d) an opening in the neck bridge through which the coils are inserted into the aneurysm sac; and (e) a closure mechanism which closes the opening after the coils have been inserted into the aneurysm sac. Alternatively, a device to occlude an aneurysm can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is at least 20% larger than the diameter of the aneurysm neck; (d) an opening in the neck bridge through which the coils are inserted into the aneurysm sac; and (e) a remotely-activated closure mechanism which closes the opening after the coils have been inserted into the aneurysm sac.

In an embodiment, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) with a radially-more-constrained first configuration and a radially-less-constrained second configuration, wherein the neck bridge is inserted into the aneurysm sac in its first configuration and self-expands into its second configuration; wherein the neck bridge has a bowl, cup, hemispherical, and/or paraboloidal shape in its second configuration; and wherein the neck bridge has a diameter which is greater than the diameter of the aneurysm neck in its second configuration; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a paraboloid-shaped neck bridge (e.g. stent, lattice, mesh, or framework) which expands (e.g. self-expands) within an aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a shaped neck bridge (e.g. stent, lattice, mesh, or framework) which is inserted into the aneurysm sac, is expanded (e.g. self-expands) into a substantially ellipsoidal shape within the aneurysm sac, and then is collapsed into a double-layer, upward-opening, bowl shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In one possible embodiment, a “bowl-of-spaghetti” type aneurysm occlusion device can comprise: (a) a catheter; (b) one or more embolic coils which are inserted through the catheter into an aneurysm sac; (c) a upwardly-convex neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type intrasacular aneurysm occlusion device can comprise: (a) a longitudinal lumen (e.g. a catheter) which is configured into be inserted into a person's vasculature; (b) one or more longitudinal embolic members (e.g. embolic coils) which are configured to be delivered through the longitudinal lumen into an aneurysm sac; (c) an expandable proximal member (e.g. a bowl or cup shaped neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck; and (d) an opening in the expandable proximal member through which the longitudinal embolic members are inserted into the aneurysm sac.

A “bowl-of-spaghetti” type aneurysm occlusion device can alternatively comprise: (a) a longitudinal lumen (e.g. a catheter) which is configured into be inserted into a person's vasculature; (b) one or more longitudinal embolic members (e.g. embolic coils) which are configured to be delivered through the longitudinal lumen into an aneurysm sac; (c) an expandable proximal member (e.g. an upward-opening-convex neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck; (d) an opening in the expandable proximal member through which the longitudinal embolic members are inserted into the aneurysm sac; and (e) a closure mechanism which closes the opening after the longitudinal embolic members have been inserted into the aneurysm sac. In yet another embodiment, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) to a disk shape within the aneurysm sac, expanding to a diameter which is larger than the diameter of the aneurysm neck; (d) a (central) opening in the expandable proximal member through which the coils are inserted into the aneurysm sac; (e) an electromagnetic coil detachment (e.g. cutting) mechanism which detaches (e.g. cuts) one or more segments of embolic coils after a portion of the embolic coils has been inserted into the aneurysm sac; and (f) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member is a tubular stent which has been inverted (or everted) into a double-layer convex shape; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprise a rotatable wire or catheter which is remotely rotated by a device user.

A “bowl-of-spaghetti” type aneurysm occlusion device can also comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism comprises electromagnetic energy which is applied to fuse opening closed. Alternatively, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism is a cap, seal, and/or threaded member.

In an embodiment, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a spring or elastic member with a first configuration which does not block (or otherwise close) the opening and a second configuration which blocks (or otherwise closes) the opening, wherein the spring or elastic member is changed from its first configuration to its second configuration by transmitting a pulse of electrical current to it. In another example, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a device to occlude an aneurysm can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which reduces the size of the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) to a bowl or cup shape within the aneurysm sac, conforming to the walls of the most proximal one-third of the aneurysm sac; (d) a (central) opening in the neck bridge through which the coils are inserted into the aneurysm sac; and (e) a closure mechanism which reduces the size of the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a non-central opening in the neck bridge through which the coils are inserted into the aneurysm sac; and (e) a closure mechanism which closes the opening after the coils have been inserted into the aneurysm sac. In another example, a device to occlude an aneurysm can be embodied in: (a) a catheter; (b) one or more embolic coils which are inserted through the catheter into an aneurysm sac; (c) an upward-facing-convex neck bridge (e.g. stent, lattice, mesh, or framework) which self-expands within the aneurysm sac, expanding to a diameter which is larger than the diameter of the aneurysm neck, wherein the neck bridge further comprises a mesh, network, lattice, or radial array of wires or other stiff fibers; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) an upward-opening convex neck bridge (e.g. stent, lattice, mesh, or framework) which expands (e.g. self-expands) within an aneurysm sac, expanding to a diameter which is larger than the diameter of the aneurysm neck, wherein the neck bridge is created by tying (or otherwise radially-constraining) and then inverting a portion of a tubular mesh; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type method for occluding an aneurysm can be embodied in: (a) inserting a neck bridge (e.g. stent, lattice, mesh, or framework) into an aneurysm sac; (b) expanding the neck bridge in the aneurysm sac into a paraboloidal shape with a diameter which is wider than the diameter of the aneurysm neck; (c) inserting embolic coils into the aneurysm sac through a central opening in the neck bridge; and (d) closing the opening in the neck bridge.

In an example, a “bowl-of-spaghetti” type method for occluding an aneurysm can comprise: (a) inserting a braided neck bridge (e.g. stent, lattice, mesh, or framework) into an aneurysm sac; (b) expanding the neck bridge in the aneurysm sac of the into a convex shape with a diameter which is wider than the diameter of the aneurysm neck; (c) inserting embolic coils into the aneurysm sac through an opening in the neck bridge; and (d) closing the opening in the neck bridge. In another example, a aneurysm occlusion method can comprise: (a) inserting a neck bridge (e.g. stent, lattice, mesh, or framework) into an aneurysm sac; (b) expanding the neck bridge in the proximal half of the aneurysm sac into a hemispherical shape with a diameter which is wider than the diameter of the aneurysm neck; (c) inserting embolic coils into the aneurysm sac through an opening in the neck bridge; and (d) closing the opening in the neck bridge.

In an example, a “bowl-of-spaghetti” type method for occluding an aneurysm can comprise: (a) inserting a braided neck bridge (e.g. stent, lattice, mesh, or framework) into an aneurysm sac; (b) expanding the neck bridge into a bowl or cup shape with a diameter which is at least 20% wider than the diameter of the aneurysm neck; (c) inserting embolic coils into the aneurysm sac through an opening in the neck bridge; and (d) closing the opening in the neck bridge. In an alternative example, a device to occlude an aneurysm can comprise: (a) a catheter; (b) one or more embolic coils which are inserted through the catheter into an aneurysm sac; (c) a 3D-printed neck bridge (e.g. stent, lattice, mesh, or framework) which expands (e.g. self-expands) to a bowl or cup shape within the aneurysm sac, expanding to a diameter which is at least 20% larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a braided neck bridge (e.g. stent, lattice, mesh, or framework) which expands (e.g. self-expands) to a bowl or cup shape within the aneurysm sac, expanding to a diameter which is at least 20% larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can be embodied in: (a) a catheter; (b) one or more helical embolic coils which are inserted through the catheter into an aneurysm sac; (c) a braided neck bridge (e.g. stent, lattice, mesh, or framework) which expands (e.g. self-expands) to a bowl or cup shape within the aneurysm sac, expanding to a diameter which is at least 20% larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a braided, knitted, or woven neck bridge (e.g. stent, lattice, mesh, or framework) which expands (e.g. self-expands) within an aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type intrasacular aneurysm occlusion device can be embodied in: (a) a catheter; (b) one or more helical embolic coils which are inserted through the catheter into an aneurysm sac; (c) a cross-sectionally-asymmetric neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a knitted, woven, or braided neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a multi-layer, concave neck bridge (e.g. stent, lattice, mesh, or framework) which expands (e.g. self-expands) within the aneurysm sac, expanding to a diameter which is larger than the maximum diameter of the aneurysm neck; (d) a (central) opening in the neck bridge through which the coils are inserted into the aneurysm sac; and (e) an electromagnetic closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, framework, lattice, or mesh) which is inserted into the aneurysm sac in a first shape, then is expanded (e.g. self-expands) within the aneurysm sac into a spherical second shape, and then is compressed and/or folded into a hemispherical third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In an alternative example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) into a bowl or cup shape within the aneurysm sac, expanding to a diameter which is larger than the maximum diameter of the aneurysm neck; (d) a (central) opening in the neck bridge through which the coils are inserted into the aneurysm sac; and (e) a chemical closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck, wherein the neck bridge is made from a plurality of braided filaments; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck, wherein the neck bridge further comprises a radiopaque marker at its distal end; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac, expanding to a diameter which is larger than the diameter of the aneurysm neck, wherein the neck bridge or stent expands in a radial-expansion plane which is parallel to the plane of the circumference of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self expands) to a bowl or cup shape within the proximal half of the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within an aneurysm sac, expanding into a bowl or cup shape with a diameter which is larger than the diameter of the aneurysm neck, wherein the neck bridge has a proximal portion with a first porosity level and a distal portion with a second porosity level, and wherein the second porosity level is greater than the first porosity level; (d) a (central) opening in the neck bridge through which the coils are inserted into the aneurysm sac; and (e) a closure mechanism which closes the opening after the coils have been inserted into the aneurysm sac.

In an example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type intrasacular aneurysm occlusion device can comprise: (a) a catheter; (b) one or more embolic coils which are transported through the catheter and inserted into an aneurysm sac; (c) a neck bridge (e.g. stent, lattice, mesh, or framework) which is transported through the catheter in a radially-more-constrained first configuration, inserted into the aneurysm sac, and then expanded (e.g. self expanded) into a radially-less-constrained second bowl or cup shaped configuration, wherein the second configuration has a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

A “bowl-of-spaghetti” type aneurysm occlusion device can also comprise: (a) a catheter; (b) one or more embolic coils with a first cross-sectional shape which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac, wherein the opening has the first cross-sectional shape when it is open and a second cross-sectional shape when it is closed; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a pull-cord or pull-wire closure mechanism which closes the opening after the coils have been inserted into the aneurysm sac, wherein the closure mechanism closes the opening when a device user pulls on a cord or wire.

In one possible embodiment, a device to occlude an aneurysm can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) an elastic ring which closes the opening after the coils have been inserted into the aneurysm sac. In yet another embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a circular opening in the neck bridge through which the coils are inserted into the aneurysm sac; and (e) a closure mechanism which closes the opening after the coils have been inserted into the aneurysm sac.

In an example, a device to occlude an aneurysm can be embodied in: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a opening in the neck bridge through which the coils are inserted into the aneurysm sac, wherein the opening is closer to the circumference of the neck bridge than to its central axis; and (e) a closure mechanism which closes the opening after the coils have been inserted into the aneurysm sac. In another example, a “bowl-of-spaghetti” type intrasacular aneurysm occlusion device can comprise: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a plurality of openings in the neck bridge through which the coils are inserted into the aneurysm sac; and (e) one or more closure mechanisms which close the plurality of openings after the coils have been inserted into the aneurysm sac.

In an embodiment, a “bowl-of-spaghetti” type aneurysm occlusion device can comprise: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an off-center opening in the neck bridge through which the coils are inserted into the aneurysm sac; and (e) a closure mechanism which closes the opening after the coils have been inserted into the aneurysm sac. Alternatively, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) a closure mechanism which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) a closure mechanism which detaches the catheter from the neck bridge and closes the opening after coils have been inserted into the aneurysm sac. In another example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter protrudes into the aneurysm sac.

In an example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a first diameter, wherein the aneurysm neck has a second diameter, wherein the aneurysm sac has a maximum third diameter in a plane which is parallel to the aneurysm neck, and wherein the first diameter is at least 125% of the second diameter and at least 80% of the third diameter; (d) an opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the diameter of the aneurysm neck; (d) an opening in the neck bridge through which the coils are inserted into the aneurysm sac; and (e) a remotely-activated closure mechanism which closes the opening after the coils have been inserted into the aneurysm sac.

In one possible embodiment, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) bowl or cup shape with a maximum first diameter, wherein the aneurysm neck has a maximum second diameter, wherein the aneurysm sac and a maximum third diameter, wherein the first diameter is between 125% and 300% of the size of the second diameter; and wherein the first diameter is between 75% and 100% of the size of the third diameter; (d) an opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) with a radially-more-constrained first configuration and a radially-less-constrained second configuration; wherein the neck bridge is inserted into the aneurysm sac in its first configuration and then expanded into its second configuration; wherein the neck bridge has a bowl, cup, hemispherical, and/or paraboloidal shape in its second configuration; and wherein the neck bridge has a diameter which is greater than 90% of the maximum diameter of the aneurysm sac in its second configuration; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can be embodied in: (a) a catheter; (b) one or more embolic coils which are inserted through the catheter into an aneurysm sac; (c) a resilient neck bridge (e.g. stent, lattice, mesh, or framework) which expands (e.g. self-expands) within an aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can comprise: (a) a catheter; (b) one or more embolic coils which are inserted through the catheter into an aneurysm sac; (c) a toroidal neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type intrasacular aneurysm occlusion device can comprise: (a) a catheter; (b) one or more embolic coils which are inserted through the catheter into an aneurysm sac; (c) an arcuate neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a device to occlude an aneurysm can comprise: (a) a longitudinal lumen (e.g. a catheter); (b) one or more longitudinal embolic members (e.g. embolic coils) which are inserted into an aneurysm sac; (c) an expandable proximal member (e.g. a downwardly-convex neck bridge) which is expanded (e.g. self-expanded) within a proximal portion the aneurysm sac to a diameter which is larger than the diameter of the aneurysm neck; (d) an opening in the expandable proximal member through which the longitudinal embolic members are inserted into the aneurysm sac; and (e) a closure mechanism which closes the opening after the longitudinal embolic members have been inserted into the aneurysm sac.

In an example, a “bowl-of-spaghetti” type intrasacular aneurysm occlusion device can comprise: (a) a longitudinal lumen (e.g. a catheter); (b) one or more longitudinal embolic members (e.g. embolic coils) which are inserted into an aneurysm sac; (c) an expandable proximal member (e.g. an upward-opening-convex neck bridge) which is expanded (e.g. self-expanded) within a proximal portion the aneurysm sac to a diameter which is larger than the diameter of the aneurysm neck; (d) an opening in the expandable proximal member through which the longitudinal embolic members are inserted into the aneurysm sac; and (e) a closure mechanism which closes the opening after the longitudinal embolic members have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) to a disk shape within the aneurysm sac, expanding to a diameter which is larger than the diameter of the aneurysm neck; (d) a (central) opening in the expandable proximal member through which the coils are inserted into the aneurysm sac; and (e) a closure mechanism which closes the opening after the coils have been inserted into the aneurysm sac.

In an embodiment, a device to occlude an aneurysm can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a drawstring, elastic ring, loop, and/or tie-off In an alternative example, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a moveable plug or valve.

A “bowl-of-spaghetti” type aneurysm occlusion device can alternatively comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a clasp, clip, or snap. In another example, a device to occlude an aneurysm can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a spring or elastic member with a first configuration which does not block (or otherwise close) the opening and a second configuration which blocks (or otherwise closes) the opening.

In an example, a “bowl-of-spaghetti” type intrasacular aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism has a first configuration which does not block (or otherwise close) the opening and a second configuration which blocks (or otherwise closes) the opening, wherein the closure mechanism is changed from its first configuration to its second configuration by stopping the transmission of electrical current to it.

A “bowl-of-spaghetti” type aneurysm occlusion device can also comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member has a toroidal shape, bowl shape, convex lens shape, cup shape, paraboloid (of revolution) shape, inverted umbrella shape, and/or hemispherical shape after it has been expanded; (d) a (central) opening in the neck bridge through which the coils are inserted into the aneurysm sac; and (e) a closure mechanism which closes the opening after the coils have been inserted into the aneurysm sac. In an example, Arnold can see a bowl of spaghetti and say “Vista La Pasta, Baby.” In an example, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is at least 20% larger than the diameter of the aneurysm neck; (d) a (central) opening in the neck bridge through which the coils are inserted into the aneurysm sac; and (e) a closure mechanism which reduces the size of the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which reduces the size of the opening after the coils have been inserted into the aneurysm sac, wherein the closure mechanism is remotely activated from a location outside the person's body by a device user. In another example, a “bowl-of-spaghetti” type intrasacular aneurysm occlusion device can comprise: (a) a catheter; (b) one or more longitudinal embolic members (e.g. embolic coils) which are inserted into an aneurysm sac; (c) an expandable upward-opening-convex neck bridge (e.g. stent, lattice, mesh, or framework) which expands (e.g. self-expands) within the aneurysm sac, expanding to a diameter which is larger than the diameter of the aneurysm neck; (d) a (central) opening in the neck bridge through which the longitudinal embolic members are inserted into the aneurysm sac; and (e) a closure mechanism which closes the opening after the longitudinal embolic members have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) an upward-facing-convex neck bridge self-expands within the aneurysm sac, expanding to a diameter which is larger than the diameter of the aneurysm neck, wherein the neck bridge is made from a metal; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) and inflatable member (e.g. balloon); (d) a multi-layer, upward-facing-convex, neck bridge (e.g. stent, lattice, mesh, or framework) which is expanded by the inflatable member within the aneurysm sac, expanding to a diameter which is larger than the diameter of the aneurysm neck; (e) a (central) opening in the neck bridge through which the coils are inserted into the aneurysm sac; and (f) a closure mechanism which closes the opening after the coils have been inserted into the aneurysm sac.

In an embodiment, a “bowl-of-spaghetti” type method for occluding an aneurysm can comprise: (a) inserting a neck bridge (e.g. stent, lattice, mesh, or framework) into an aneurysm sac; (b) expanding the neck bridge in the proximal half of the aneurysm sac into a paraboloidal shape with a diameter which is wider than the diameter of the aneurysm neck; (c) inserting embolic coils into the aneurysm sac through a central opening in the neck bridge; and (d) closing the opening in the neck bridge. In another example, a “bowl-of-spaghetti” type method for occluding an aneurysm can be embodied in: (a) inserting a braided neck bridge (e.g. stent, lattice, mesh, or framework) into an aneurysm sac; (b) expanding the neck bridge in the aneurysm sac of the into a hemispherical shape with a diameter which is wider than the diameter of the aneurysm neck; (c) inserting embolic coils into the aneurysm sac through an opening in the neck bridge; and (d) closing the opening in the neck bridge.

In an example, a aneurysm occlusion method can comprise: (a) inserting a neck bridge (e.g. stent, lattice, mesh, or framework) into an aneurysm sac; (b) expanding the neck bridge into a bowl or cup shape with a diameter which is at least 20% wider than the diameter of the aneurysm neck; (c) inserting embolic coils into the aneurysm sac through an opening in the neck bridge; and (d) closing the opening in the neck bridge. In an alternative example, a “bowl-of-spaghetti” type method for occluding an aneurysm can be embodied in: (a) inserting a braided neck bridge (e.g. stent, lattice, mesh, or framework) into an aneurysm sac; (b) expanding the neck bridge into a bowl or cup shape with a diameter which is between 90% and 100% of the maximum diameter of the aneurysm sac; (c) inserting embolic coils into the aneurysm sac through an opening in the neck bridge; and (d) closing the opening in the neck bridge. In another example, a “bowl-of-spaghetti” type aneurysm occlusion device can comprise: (a) a catheter; (b) one or more embolic coils which are inserted through the catheter into an aneurysm sac; (c) a braided multi-layer neck bridge (e.g. stent, lattice, mesh, or framework) which expands (e.g. self-expands) to a bowl or cup shape within the aneurysm sac, expanding to a diameter which is at least 20% larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, an intrasacular aneurysm occlusion device can comprise: (a) a catheter; (b) one or more embolic coils which are inserted through the catheter into an aneurysm sac; (c) a braided neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In another example, an intrasacular aneurysm occlusion device can comprise: (a) a catheter; (b) a neck bridge (e.g. stent, lattice, mesh, or framework) which expands (e.g. self-expands) to a bowl or cup shape within the aneurysm sac; (c) an opening in the neck bridge; (d) liquid embolic material which is inserted into the aneurysm sac through the opening and then congeals within the aneurysm sac; and (e) a closure mechanism which closes the opening after the liquid embolic material has been inserted into the aneurysm sac. In another example, a aneurysm occlusion device can comprise: (a) a catheter; (b) one or more embolic coils which are inserted through the catheter into an aneurysm sac; (c) a braided Nitinol neck bridge (e.g. stent, framework, lattice, or mesh) which is inserted into the aneurysm sac in a first shape, then is expanded (e.g. self-expands) within the aneurysm sac into a spherical second shape, and then is compressed and/or folded into a hemispherical third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

A “bowl-of-spaghetti” type intrasacular aneurysm occlusion device can also comprise: (a) a catheter; (b) one or more embolic coils which are inserted through the catheter into an aneurysm sac; (c) a braided, knitted, or woven neck bridge (e.g. stent, lattice, mesh, or framework) which expands (e.g. self-expands) within the aneurysm sac, expanding to a diameter which is larger than the diameter of the aneurysm neck; (d) an opening in the neck bridge through which the coils are inserted into the aneurysm sac; and (e) a closure mechanism which closes the opening after the coils have been inserted into the aneurysm sac. In another example, a device to occlude an aneurysm can comprise: (a) a catheter; (b) one or more embolic coils which are inserted through the catheter into an aneurysm sac; (c) a double-layer neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can be embodied in: (a) a catheter; (b) one or more embolic coils which are inserted through the catheter into an aneurysm sac; (c) a multi-layer neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a multi-layer, convex neck bridge (e.g. stent, lattice, mesh, or framework) which expands (e.g. self-expands) within the aneurysm sac, expanding to a diameter which is larger than the maximum diameter of the aneurysm neck; (d) a (central) opening in the neck bridge through which the coils are inserted into the aneurysm sac; and (e) an electromagnetic closure mechanism which closes the opening after the coils have been inserted into the aneurysm sac. In yet another embodiment, a “bowl-of-spaghetti” type intrasacular aneurysm occlusion device can be embodied in: (a) a catheter; (b) one or more embolic coils which are inserted through the catheter into an aneurysm sac; (c) a neck bridge (e.g. stent, framework, lattice, or mesh) which is inserted into the aneurysm sac in a first shape, then is expanded (e.g. self-expands) within the aneurysm sac into an ellipsoidal second shape, and then is compressed and/or folded into a bowl or cup third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a concave shape within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a device to occlude an aneurysm can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a prolate or oblate spheroidal shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In one possible embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck, wherein the neck bridge further comprises one or more portions made with bismuth; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) within the aneurysm sac at a location from which it contacts the aneurysm sac at the sac's maximum circumference; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self expands) to a bowl or cup shape within the most proximal one-third of the volume of the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac. In another example, a device to occlude an aneurysm can comprise: (a) a catheter; (b) one or more embolic coils which are inserted through the catheter into an aneurysm sac, wherein the coils have an average first cross-sectional area; (c) a neck bridge (e.g. stent, lattice, mesh, or framework) which expands (e.g. self-expands) to a bowl or cup shape within the aneurysm sac, expanding to a diameter which is larger than the diameter of the aneurysm neck; (d) a (central) opening in the neck bridge through which the coils are inserted into the aneurysm sac, wherein the opening has a second cross-sectional area when open and a third cross-sectional area when closed, wherein the second cross-sectional area is at least 25% greater than the first cross-sectional area, and wherein the first cross-sectional area is at least 25% greater than the third cross-sectional area; and (e) a closure mechanism which closes the opening after the coils have been inserted into the aneurysm sac.

A “bowl-of-spaghetti” type aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) within an aneurysm sac, expanding into a bowl or cup shape with a diameter which is larger than the diameter of the aneurysm neck, wherein the proximal half of the neck bridge has a first porosity level, wherein the distal half of the neck bridge has a second porosity level, and wherein the second porosity level is greater than the first porosity level; (d) a (central) opening in the neck bridge through which the coils are inserted into the aneurysm sac; and (e) a closure mechanism which closes the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which is inserted into the aneurysm sac, is expanded (e.g. self-expands) into a substantially spherical shape within the aneurysm sac, and then is collapsed into a double-layer, upward-opening, hemispherical shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type intrasacular aneurysm occlusion device can comprise: (a) a catheter; (b) one or more embolic coils which are transported through the catheter and inserted into an aneurysm sac; (c) a neck bridge (e.g. stent, lattice, mesh, or framework) which is transported through the catheter in a radially-more-constrained first configuration, inserted into the aneurysm sac, and then expanded (e.g. self expanded) from the first configuration to a radially-less-constrained second ellipsoidal configuration, wherein the second configuration has a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type aneurysm occlusion device can be embodied in: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) a spring which closes the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a (central) opening in the neck bridge through which the coils are inserted into the aneurysm sac; and (e) an electrical energy closure mechanism which closes the opening after the coils have been inserted into the aneurysm sac.

A “bowl-of-spaghetti” type intrasacular aneurysm occlusion device can also comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a hexagonal opening in the neck bridge through which the coils are inserted into the aneurysm sac; and (e) a closure mechanism which closes the opening after the coils have been inserted into the aneurysm sac. Alternatively, a “bowl-of-spaghetti” type aneurysm occlusion device can comprise: (a) a catheter; (b) one or more helical embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a opening in the neck bridge through which the coils are inserted into the aneurysm sac, wherein the opening is closer to the cross-sectional perimeter of the neck bridge than to the central axis of the neck bridge; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) a quadrilateral opening in the neck bridge through which the coils are inserted into the aneurysm sac; and (e) a closure mechanism which closes the opening after the coils have been inserted into the aneurysm sac. In another example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in base of the neck bridge through which the coils are inserted into the aneurysm sac; (e) a closure mechanism which closes the opening after the coils have been inserted into the aneurysm sac; and (f) a detachment mechanism which cuts or otherwise detaches segments of embolic coils.

In an embodiment, a “bowl-of-spaghetti” type aneurysm occlusion device can be embodied in: (a) a catheter; (b) one or more embolic coil which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) a closure mechanism which detaches a segment of embolic coil, detaches the catheter from the neck bridge, and closes the opening. In an alternative example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which is larger than the maximum diameter of the aneurysm neck; (d) an opening in the neck bridge through which the catheter extends and through which coils are inserted into the aneurysm sac; and (e) a closure mechanism which detaches the catheter from the opening and closes the opening.

In an example, an intrasacular aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) bowl or cup shape with a maximum first diameter, wherein the aneurysm neck has a maximum second diameter, wherein the aneurysm sac and a maximum third diameter, wherein the first diameter is greater than the second diameter and less than the third diameter; (d) an opening in the neck bridge through which the coils are inserted into the aneurysm sac; and (e) a closure mechanism which closes the opening after the coils have been inserted into the aneurysm sac. In another example, an intrasacular aneurysm occlusion device can comprise: (a) a catheter; (b) a neck bridge (e.g. stent, lattice, mesh, or framework) which expands (e.g. self-expands) to a bowl or cup shape within the aneurysm sac; (c) an opening in the neck bridge; (d) a plurality of compressible embolic members (e.g. hydrogels or microsponges) which are inserted into the aneurysm sac through the opening; and (e) a closure mechanism which closes the opening after the liquid embolic material has been inserted into the aneurysm sac. In an example, an aneurysm occlusion device can comprise: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a first diameter, wherein the aneurysm neck has a second diameter, wherein the aneurysm sac has a maximum third diameter in a plane which is parallel to the aneurysm neck, and wherein the first diameter is at least 150% of the second diameter and at least 95% of the third diameter; (d) an opening in the neck bridge through which the coils are inserted into the aneurysm sac; and (e) a closure mechanism which closes the opening after the coils have been inserted into the aneurysm sac.

In one possible embodiment, a “bowl-of-spaghetti” type intrasacular aneurysm occlusion device can be embodied in: (a) a catheter; (b) one or more embolic coils which are inserted through the catheter into an aneurysm sac; (c) a neck bridge (e.g. stent, lattice, mesh, or framework) which expands (e.g. self-expands) to a bowl or cup shape within the aneurysm sac, expanding to a diameter which at least 90% of the maximum diameter of the aneurysm sac in a plane parallel to the aneurysm neck; (d) an opening in the neck bridge through which the coils are inserted into the aneurysm sac; and (e) a remotely-activated closure mechanism which closes the opening after the coils have been inserted into the aneurysm sac. In another example, a device to occlude an aneurysm can comprise: (a) a catheter; (b) one or more embolic coils which are inserted through the catheter into an aneurysm sac; (c) a neck bridge (e.g. stent, lattice, mesh, or framework) which expands (e.g. self-expands) bowl or cup shape with a maximum first diameter, wherein the aneurysm neck has a maximum second diameter, wherein the aneurysm sac and a maximum third diameter, wherein the first diameter is between 110% and 200% of the size of the second diameter; and wherein the first diameter is between 85% and 100% of the size of the third diameter; (d) an opening in the neck bridge through which the coils are inserted into the aneurysm sac; and (e) a closure mechanism which closes the opening after the coils have been inserted into the aneurysm sac.

In an example, a “bowl-of-spaghetti” type intrasacular aneurysm occlusion device can comprise: (a) a catheter; (b) one or more embolic coils which are inserted through the catheter into an aneurysm sac; (c) a Nitinol neck bridge (e.g. stent, framework, lattice, or mesh) which is inserted into the aneurysm sac in a first shape, then expanded (e.g. self-expands) within the aneurysm sac into a spherical second shape, and then compressed and/or folded into a hemispherical third shape with a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. Alternatively, a “bowl-of-spaghetti” type aneurysm occlusion device can comprise: (a) a catheter; (b) one or more embolic coils which are inserted through the catheter into an aneurysm sac; (c) a shaped neck bridge (e.g. stent, lattice, mesh, or framework) which is inserted into the aneurysm sac, is expanded (e.g. self-expands) into a substantially ellipsoidal shape within the aneurysm sac, and then is collapsed into a double-layer, upward-opening, substantially paraboloidal shape which substantially covers the inside of the aneurysm neck; (d) a (central) opening in the neck bridge through which the coils are inserted into the aneurysm sac; and (e) a closure mechanism which closes the opening after the coils have been inserted into the aneurysm sac.

In an embodiment, a “bowl-of-spaghetti” type aneurysm occlusion device can be embodied in: (a) a catheter; (b) one or more embolic coils which are inserted through the catheter into an aneurysm sac; (c) a toroidal neck bridge (e.g. stent, lattice, mesh, or framework) which is expanded within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac.

An aneurysm occlusion device can also comprise: (a) a longitudinal lumen (e.g. a catheter) which is configured into be inserted into a person's vasculature; (b) one or more longitudinal embolic members (e.g. embolic coils) which are configured to be delivered through the longitudinal lumen into an aneurysm sac; (c) an expandable proximal member (e.g. a bowl or cup shaped neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck after the expandable proximal member has been expanded; (d) an opening in the expandable proximal member through which the longitudinal embolic members are inserted into the aneurysm sac; and (e) a closure mechanism which closes the opening after the longitudinal embolic members have been inserted into the aneurysm sac. In another example, an intrasacular aneurysm occlusion device can comprise: (a) a catheter; (b) a neck bridge (e.g. stent, lattice, mesh, or framework) which expands (e.g. self-expands) to a bowl or cup shape within the aneurysm sac; (c) an opening in the neck bridge; (d) one or more “string-of-pearls” type longitudinal embolic members (e.g. microsponges connected by filaments) which are inserted into the aneurysm sac through the opening; and (e) a closure mechanism which closes the opening after the liquid embolic material has been inserted into the aneurysm sac. In another example, an aneurysm occlusion device can comprise: (a) a longitudinal lumen (e.g. a catheter); (b) one or more longitudinal embolic members (e.g. embolic coils) which are inserted into an aneurysm sac; (c) an expandable proximal member (e.g. an upwardly-convex neck bridge) which is expanded (e.g. self-expanded) within a proximal portion the aneurysm sac to a diameter which is larger than the diameter of the aneurysm neck; (d) an opening in the expandable proximal member through which the longitudinal embolic members are inserted into the aneurysm sac; and (e) a closure mechanism which closes the opening after the longitudinal embolic members have been inserted into the aneurysm sac.

In an example, a “bowl-of-spaghetti” type aneurysm occlusion device can be embodied in: (a) a longitudinal lumen (e.g. a catheter) which is configured into be inserted into a person's vasculature; (b) one or more longitudinal embolic members (e.g. embolic coils) which are configured to be delivered through the longitudinal lumen into an aneurysm sac; (c) an expandable proximal member (e.g. an upward-opening-convex neck bridge) which is configured to be inserted and expanded (e.g. self-expanded) within a proximal portion the aneurysm sac to a diameter which is larger than the diameter of the aneurysm neck, wherein the expandable proximal member spans the aneurysm neck; and (d) an opening in the expandable proximal member through which the longitudinal embolic members are inserted into the aneurysm sac. In an alternative example, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member is a spherical stent which has been collapsed or folded into a hemispherical shape; (d) a (central) opening in the neck bridge through which the coils are inserted into the aneurysm sac; and (e) a closure mechanism which closes the opening after the coils have been inserted into the aneurysm sac.

In an embodiment, a device to occlude an aneurysm can be embodied in: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a pull cord or pull wire.

A “bowl-of-spaghetti” type intrasacular aneurysm occlusion device can alternatively comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism is an adhesive which is released into the opening. In another example, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises application of electromagnetic energy to shape-memory material.

In an embodiment, a “bowl-of-spaghetti” type intrasacular aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the closure mechanism further comprises a spring or elastic member with a first configuration which does not block (or otherwise close) the opening and a second configuration which blocks (or otherwise closes) the opening, wherein the spring or elastic member is changed from its first configuration to its second configuration by the transmission of electromagnetic energy. In another example, a device to occlude an aneurysm can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a closure mechanism which closes the opening after the coils have been inserted into the aneurysm sac, wherein the opening is held open by the transmission of electromagnetic energy and closes when the transmission of electromagnetic energy is stopped.

In an example, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck, wherein the expandable proximal member has an annular shape, cylindrical shape doughnut shape, ring shape, tire shape, or wheel shape after it has been expanded; (d) a (central) opening in the neck bridge through which the coils are inserted into the aneurysm sac; and (e) a closure mechanism which closes the opening after the coils have been inserted into the aneurysm sac.

In an example, a “bowl-of-spaghetti” type aneurysm occlusion device can comprise: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is at least 50% larger than the diameter of the aneurysm neck; (d) a (central) opening in the neck bridge through which the coils are inserted into the aneurysm sac; and (e) a closure mechanism which reduces the size of the opening after the coils have been inserted into the aneurysm sac. In another example, a “bowl-of-spaghetti” type aneurysm occlusion device can be embodied in: (a) a catheter; (b) one or more embolic coils which are inserted through the catheter into an aneurysm sac; (c) an expandable proximal member (e.g. neck bridge) which expands (e.g. self-expands) within the aneurysm sac, expanding to a diameter which is larger than the diameter of the aneurysm neck; (d) a (central) opening in the neck bridge through which the coils are inserted into the aneurysm sac; and (e) a one-way valve which allows coils to be inserted through the opening into the aneurysm sac, but does not allow coils to escape out of the aneurysm sac.

A “bowl-of-spaghetti” type intrasacular aneurysm occlusion device can alternatively comprise: (a) a catheter; (b) one or more embolic coils which are inserted through the catheter into an aneurysm sac; (c) an inflatable toroidal neck bridge (e.g. stent, lattice, mesh, or framework) which is expanded within an aneurysm sac, expanding to a diameter which is larger than the maximum diameter of the aneurysm neck; (d) a (central) opening in the neck bridge through which the coils are inserted into the aneurysm sac; and (e) a closure mechanism which closes the opening after the coils have been inserted into the aneurysm sac. In yet another embodiment, a “bowl-of-spaghetti” type aneurysm occlusion device can comprise: (a) a catheter; (b) one or more embolic coils which are inserted through the catheter into an aneurysm sac; (c) an upward-facing-convex neck bridge self-expands within the aneurysm sac, expanding to a diameter which is larger than the diameter of the aneurysm neck, wherein the neck bridge is made from a polymer; (d) a (central) opening in the neck bridge through which the coils are inserted into the aneurysm sac; and (e) a closure mechanism which closes the opening after the coils have been inserted into the aneurysm sac.

In one possible embodiment, a “bowl-of-spaghetti” type method for occluding an aneurysm can be embodied in: (a) inserting a braided neck bridge (e.g. stent, lattice, mesh, or framework) into an aneurysm sac; (b) allowing the neck bridge to self-expand into a spherical or ellipsoidal first shape; (c) axially contracting, compressing, and/or folding the neck bridge from the first shape into a double-layer hemispherical second shape; (d) inserting embolic coils into the aneurysm sac through an opening in the neck bridge; and (e) closing the opening in the neck bridge. In another example, a “bowl-of-spaghetti” type method for occluding an aneurysm can comprise: (a) inserting a neck bridge (e.g. stent, lattice, mesh, or framework) into an aneurysm sac; (b) expanding the neck bridge in the aneurysm sac into a convex shape with a diameter which is wider than the diameter of the aneurysm neck; (c) inserting embolic coils into the aneurysm sac through an opening in the neck bridge; and (d) closing the opening in the neck bridge.

In an example, a “bowl-of-spaghetti” type method for occluding an aneurysm can comprise: (a) inserting a braided neck bridge (e.g. stent, lattice, mesh, or framework) into an aneurysm sac; (b) expanding the neck bridge in the aneurysm sac of the into a paraboloidal shape with a diameter which is wider than the diameter of the aneurysm neck; (c) inserting embolic coils into the aneurysm sac through an opening in the neck bridge; and (d) closing the opening in the neck bridge. In an alternative example, a “bowl-of-spaghetti” type method for occluding an aneurysm can comprise: (a) inserting a neck bridge (e.g. stent, lattice, mesh, or framework) into an aneurysm sac; (b) expanding the neck bridge into a bowl or cup shape with a diameter which is wider than the diameter of the aneurysm neck; (c) inserting embolic coils into the aneurysm sac through an opening in the neck bridge; and (d) closing the opening in the neck bridge. In another example, a aneurysm occlusion method can be embodied in: (a) inserting a neck bridge (e.g. stent, lattice, mesh, or framework) into an aneurysm sac; (b) releasing the neck bridge to self expand into a bowl or cup shape with a diameter which is wider than the diameter of the aneurysm neck; (c) inserting embolic coils into the aneurysm sac through an opening in the neck bridge; and then (d) closing the opening in the neck bridge.

In an example, a “bowl-of-spaghetti” type intrasacular aneurysm occlusion device can comprise: a longitudinal lumen which is configured into be inserted into a person's vasculature; one or more longitudinal embolic members which are configured to be delivered through the longitudinal lumen into an aneurysm sac in the person's vasculature; an expandable proximal member which is configured to be inserted into and expanded within the aneurysm sac, expanding to a diameter which is larger than the maximum diameter of the aneurysm neck; an opening in the expandable proximal member through which the longitudinal embolic members are inserted into the aneurysm sac; and a closure mechanism which closes the opening after the longitudinal embolic members have been inserted into the aneurysm sac.

In an example, a longitudinal lumen can be a catheter, longitudinal embolic members can be embolic coils, and an expandable proximal member can be a neck bridge. In an example, an expandable proximal member (e.g. neck bridge) can self expand within the aneurysm sac. In an example, an expandable proximal member (e.g. neck bridge) can expand into an upward-opening-convex shape. In an example, an expandable proximal member (e.g. neck bridge) can expand into a bowl or cup shape. In an example, an expandable proximal member (e.g. neck bridge) can be a braided or woven stent, lattice, mesh, and/or framework. In an example, an expandable proximal member (e.g. neck bridge) can be a 3D printed stent, lattice, mesh, and/or framework.

In an example, an expandable proximal member (e.g. neck bridge) can be a multi-layer neck bridge which is made by folding and/or compressing a generally spherical or ellipsoidal stent, lattice, mesh, or framework into a multi-layer bowl or cup shaped stent, lattice, mesh, or framework. In an example, an expandable proximal member (e.g. neck bridge) can be a multi-layer neck bridge which is made by folding and/or compressing a generally spherical or ellipsoidal stent, lattice, mesh, or framework within the aneurysm sac. In an example, an expandable proximal member (e.g. neck bridge) can be a multi-layer neck bridge which is made by folding, twisting, and/or inverting a generally tubular stent, lattice, mesh, or framework into a multi-layer bowl or cup shaped stent, lattice, mesh, or framework.

In an example, an expandable proximal member (e.g. neck bridge) can expand to a diameter which is at least 10% larger than the maximum diameter of the aneurysm neck. In an example, an expandable proximal member (e.g. neck bridge) can expand to a diameter which is at least 85% of the maximum diameter of the aneurysm sac. In an example, an expandable proximal member (e.g. neck bridge) can expand to a diameter which is at least 10% larger than the maximum diameter of the aneurysm neck and at least 85% of the maximum diameter of the aneurysm sac. In an example, a longitudinal lumen (e.g. catheter) can connect to and/or protrude into the opening in the expandable proximal member.

In an example, a closure mechanism can be remotely-activated by the application of electromagnetic energy. In an example, a closure mechanism can close the opening in the expandable proximal member and detach an embolic longitudinal member which has been inserted into the aneurysm sac. In an example, a closure mechanism can close the opening in the expandable proximal member, detach an embolic longitudinal member which has been inserted into the aneurysm sac, and detach the longitudinal lumen from the expandable proximal member so that the longitudinal lumen can be withdrawn from the person's vasculature.

In an example, a “bowl-of-spaghetti” type intrasacular aneurysm occlusion device can comprise: a catheter which is inserted into a person's vasculature; one or more embolic coils which are delivered through the catheter into an aneurysm sac; a neck bridge which expands into a bowl or cup shape within the aneurysm sac, expanding to a diameter which is larger than the maximum diameter of the aneurysm neck; an opening in the neck bridge through which the coils are inserted into the aneurysm sac; and a closure mechanism which closes the opening after the coils have been inserted into the aneurysm sac.

In an example, a “bowl-of-spaghetti” type method of occluding an aneurysm can comprise: inserting a catheter into a person's vasculature; inserting a neck bridge on or through the catheter into an aneurysm sac in the person's vasculature; expanding the neck bridge into a bowl or cup shape within the aneurysm sac, wherein the neck bridge is expanded to a diameter which is larger than the maximum diameter of the aneurysm neck; inserting one or more embolic coils into the aneurysm through the catheter and through an opening in the neck bridge; closing the opening in the neck bridge; and withdrawing the catheter from the person's vasculature.

I claim:

1. A “bowl-of-spaghetti” type intrasacular aneurysm occlusion device comprising:
a longitudinal lumen which is configured into be inserted into a person's vasculature;
one or more longitudinal embolic members which are configured to be delivered through the longitudinal lumen into an aneurysm sac in the person's vasculature;
an expandable proximal member which is configured to be inserted into and expanded within the aneurysm sac, expanding to a diameter which is larger than the maximum diameter of the aneurysm neck;
an opening in the expandable proximal member through which the longitudinal embolic members are inserted into the aneurysm sac; and
a closure mechanism which closes the opening after the longitudinal embolic members have been inserted into the aneurysm sac.

2. The device in claim 1 wherein the longitudinal lumen is a catheter, the longitudinal embolic members are embolic coils, and the expandable proximal member is a neck bridge.

3. The device in claim 1 wherein the expandable proximal member self expands within the aneurysm sac.

4. The device in claim 1 wherein the expandable proximal member expands into an upward-opening-convex shape.

5. The device in claim 1 wherein the expandable proximal member expands into a bowl or cup shape.

6. The device in claim 1 wherein the expandable proximal member is a neck bridge.

7. The device in claim 6 wherein the neck bridge is a braided or woven stent, lattice, mesh, and/or framework.

8. The device in claim 6 wherein the neck bridge is a 3D printed stent, lattice, mesh, and/or framework.

9. The device in claim 6 wherein the neck bridge is a multi-layer neck bridge which is made by folding and/or compressing a generally spherical or ellipsoidal stent, lattice, mesh, or framework into a multi-layer bowl or cup shaped stent, lattice, mesh, or framework.

10. The device in claim 6 wherein the neck bridge is a multi-layer neck bridge which is made by folding and/or compressing a generally spherical or ellipsoidal stent, lattice, mesh, or framework within the aneurysm sac.

11. The device in claim 6 wherein the neck bridge is a multi-layer neck bridge which is made by folding, twisting, and/or inverting a generally tubular stent, lattice, mesh, or framework into a multi-layer bowl or cup shaped stent, lattice, mesh, or framework.

12. The device in claim 1 wherein the expandable proximal member expands to a diameter which is at least 10% larger than the maximum diameter of the aneurysm neck.

13. The device in claim 1 wherein the expandable proximal member expands to a diameter which is at least 85% of the maximum diameter of the aneurysm sac.

14. The device in claim 1 wherein the expandable proximal member expands to a diameter which is at least 10% larger than the maximum diameter of the aneurysm neck and at least 85% of the maximum diameter of the aneurysm sac.

15. The device in claim 1 wherein the longitudinal lumen connects to and/or protrudes into the opening in the expandable proximal member.

16. The device in claim 1 wherein the closure mechanism is remotely-activated by the application of electromagnetic energy.

17. The device in claim 1 wherein the closure mechanism closes the opening in the expandable proximal member and detaches an embolic longitudinal member which has been inserted into the aneurysm sac.

18. The device in claim 1 wherein the closure mechanism closes the opening in the expandable proximal member, detaches an embolic longitudinal member which has been inserted into the aneurysm sac, and detaches the longitudinal lumen from the expandable proximal member so that the longitudinal lumen can be withdrawn from the person's vasculature.

19. A “bowl-of-spaghetti” type intrasacular aneurysm occlusion device comprising:
a catheter which is inserted into a person's vasculature;
one or more embolic coils which are delivered through the catheter into an aneurysm sac;
a neck bridge which expands into a bowl or cup shape within the aneurysm sac, expanding to a diameter which is larger than the maximum diameter of the aneurysm neck;
an opening in the neck bridge through which the coils are inserted into the aneurysm sac; and
a closure mechanism which closes the opening after the coils have been inserted into the aneurysm sac.

20. A “bowl-of-spaghetti” type method of occluding an aneurysm comprising:
inserting a catheter into a person's vasculature;
inserting a neck bridge on or through the catheter into an aneurysm sac in the person's vasculature;
expanding the neck bridge into a bowl or cup shape within the aneurysm sac, wherein the neck bridge is expanded to a diameter which is larger than the maximum diameter of the aneurysm neck;
inserting one or more embolic coils into the aneurysm through the catheter and through an opening in the neck bridge;
closing the opening in the neck bridge; and
withdrawing the catheter from the person's vasculature.
